(19) United States
(12) Patent Application Publication
(10) Pub. No.: US 2024/0269455 A1
(43) Pub. Date: Aug. 15, 2024

(54) FILTRATION DEVICE AND PACKAGING CONFIGURED FOR STERILIZATION

(71) Applicant: EMD Millipore Corporation, Burlington, MA (US)

(72) Inventors: Heejin Lee, Bedford, MA (US); Michael James Susienka, Sutton, MA (US); John Paul Amara, Reading, MA (US)

(21) Appl. No.: 18/411,879

(22) Filed: Jan. 12, 2024

Related U.S. Application Data
(60) Provisional application No. 63/484,002, filed on Feb. 9, 2023.

(51) Int. Cl. A61M 39/18; A61M 39/14

(52) U.S. Cl. CPC A61M 39/18; A61M 39/14; A61M 2205/75

(57) ABSTRACT

A system for sterilizing or bioburden-reducing a filter device includes a breathable microbial barrier, a filter device, and a tubing arrangement. The tubing arrangement may be coupled to at least one opening of the filter device. The tubing arrangement may include an aseptic connector, a sealable component, and a port, all of which are in fluid communication with one another. The sealable component may be disposed in the tubing arrangement between the at least one opening of the filter device and the aseptic connector. The sealable component may be reconfigurable between an open configuration, which facilitates the entry of a sterilization vapor into the tubing arrangement and the filter device when subjected to a sterilization or bioburden reduction process, and a closed configuration, where the tubing arrangement is sealed from a surrounding environment. The breathable microbial barrier may at least partially enclose the port of the tubing arrangement.

### RELATED APPLICATIONS

The present application claims the benefit of priority of U.S. Provisional Application No. 63/484,002, filed Feb. 9, 2023, the entire contents is incorporation herein by reference.

### TECHNICAL FIELD

Embodiments of the technologies disclosed herein relate to downstream filters and filtration systems useful in bioprocessing and, particularly, the filtration devices described herein are configured for sterilization.

### BACKGROUND OF THE INVENTION/DESCRIPTION OF RELATED ART

Sterilization of bioprocessing apparatuses, such as filters and filtration systems, which are often made of polymeric materials, is required before use. There are many processes for sterilization, such as steaming, ethylene oxide gas treatment, and irradiation. Gamma irradiation, such as that with a sterilizing dose of 25-40 kilograys (kGy), is a common sterilization method used for filter devices for bioprocessing. However, gamma or other radiation sterilization methods (e.g., E-beam and X-ray) may not be compatible with certain filter materials and/or one or more filter device housing materials due to the release of irradiation-induced extractables and leachables, which can be harmful to and/or damage cells and therapeutics. Moreover, polymer degradation, which can weaken the filter medium or device housing, polymer discoloration, or other filter performance degradation, can occur. For example, certain polymeric materials, such as PTFE polymers, show poor compatibility with gamma irradiation at 25-40 kGy. In such cases, steam sterilization or autoclaving (or other gas- or vapor-based methods, such as, but not limited to, ethylene oxide, chlorine dioxide, or vaporized hydrogen peroxide) can be considered alternative sterilization methods. Autoclaving is one common method to sterilize or sanitize medical devices and bioprocessing equipment, including single-use filters. In contrast to radiation-based sterilization methods, most autoclaving methods need an open pathway for saturated steam to enter the filter device and contact all internal fluid paths during an autoclaving cycle, to render sterility or bioburden reduction.

Filter devices designed for closed processing applications often have sterile-to-sterile or aseptic connecters attached thereto, which maintain their sterility (or bioburden reduction) after the devices are sterilized. Some connectors are autoclavable aseptic connectors, such as Lynx® S2S (manufactured by EMD Millipore Corporation, Burlington, MA, USA), Kleenpak® Presto (manufactured by Pall Corporation, NY, USA), Opta® SFT (marketed by Sartorius Stedim Biotech GmbH of DE), AseptiQuik® G HT (manufactured by Colder Products Company), and ReadyMate (manufactured by Cytiva). Kleenpak Presto, Opta SFT, AseptiQuik® G HT, and ReadyMate have a protective film(s) or membrane that is removed during connection. The membrane materials used in these aseptic connectors include hydrophobic polyethersulfone for Opta SFT and hydrophobic polyethersulfone with polytetrafluoroethylene (PTFE) strip for AseptiQuik® G HT.

When these connectors are attached to one or more of the ports (inlet, vent, and outlet) of a depth filter device using autoclavable tubing (silicone or thermoplastic tubing that can withstand autoclaving temperature, e.g., 123° C.), steam can permeate through these membranes. However, steam or gas permeability through these membranes may be insufficient to achieve effective autoclaving or moist heat sterilization. If the membrane gas permeability is not sufficient, the sterilant, e.g., steam or ethylene oxide (EtO), cannot fully saturate the fluid paths inside the device, which results in incomplete sterilization of the device. An autoclave cycle can include one or more pre-vacuum cycles (or vacuum pulses) to remove air prior to steam introduction to achieve the complete replacement of the air with steam (i.e., no entrapment of air within a filter device). However, if the permeability of gas through the membrane of the aseptic connector is insufficient, the housing of the filter device can become deformed or ballooned after an autoclave cycle, due to the high temperatures and pressure differential between the inside and outside of the device housing. To implement an autoclavable sterilizable depth filter device, there is a need to configure the design of the device to provide a sufficiently permeable route for steam and air during the autoclave cycle(s) while simultaneously forming an adequate sterile barrier for the device. Furthermore, the sterile barrier should be tamper-resistant or tamper-proof to reduce the risk of inadvertent or accidental breach or compromise during shipping and handling that would render the device unsuitable and/or unusable for operations that necessitate sterility. During the depth filter operation itself, a tamper-resistant or tamper-proof closure is important to ensure operator safety due to the significant operating pressures potentially reached (up to 30 pounds per square inch (psi)).

### SUMMARY OF THE INVENTION

Embodiments described herein are, e.g., closed processing filtration devices, depth filter devices, and the like, are configured for autoclaving and other gas- or vapor-based sterilization methods, such as ethylene oxide, chlorine dioxide, ozone, supercritical carbon dioxide, and vaporized hydrogen peroxide. The design is particularly useful when the filter material is not compatible with radiation-based sterilization methods (gamma, E-beam, or X-ray). Filtration devices have tubing and aseptic connectors, and one or more ports are covered or enclosed with a breathable microbial barrier. Such port(s) remain(s) open during autoclaving and can be closed after sterilization or bioburden reduction such that the sterile (or bioburden-reduced) device is closed, and/or packaged, and remains sterile (or bioburden-reduced) until use.

In an embodiment, a tubing arrangement for sterilizing a filter device may include a first tube, a second tube, and a sealable component. The first tube may be coupled to a connector of the filter device. The second tube may be coupled to an aseptic connector. The sealable component may be disposed in the tubing arrangement between the first and second tubes. The sealable component may be reconfigurable between an open configuration, which facilitates entry of a sterilization vapor into the tubing arrangement and the filter device when subjected to a sterilization process, and a closed configuration, where the tubing arrangement is sealed from a surrounding environment.

In some instances, the tubing arrangement may further include a tee fitting having a first end, a second end, and a third end, where the first end may be coupled to the first tube and the second end may be coupled to the second tube. In some further instances, the sealable component may be coupled to the third end of the tee fitting and the sealable component may be a spring actuated valve component that includes a male portion and a female portion. The female portion may be removably coupled to the male portion. When the female portion is coupled to the male portion, the sealable component may be in the open configuration. When the female portion is uncoupled from the male portion, the sealable component may be in the closed configuration. In some other instances, a third tubing may be coupled to the third end of the tee fitting and the sealable component may be a pinch clamp disposed on the third tubing.

In even some further instances, the sealable component may be a stopcock coupled to both the first tube and the second tube. In some additional instances, the sealable component may be a rotatable sleeve valve component. In some other instances, the sealable component may be a magnetic valve component.

In another embodiment, a system for sterilizing a filter device may include a filter device, a tubing arrangement, and a breathable microbial barrier. The filter device may have at least one opening. The tubing arrangement may be coupled to the at least one opening of the filter device and may include an aseptic connector, a port, and a sealable component. The port may be in fluid communication with the aseptic connector. The sealable component may be disposed in the tubing arrangement between the at least one opening of the filter device and the aseptic connector. In addition, the sealable component may be in fluid communication with the at least one opening of the filter device, the aseptic connector, and the port. The sealable component may be reconfigurable between an open configuration, which facilitates entry of a sterilization vapor into the tubing arrangement and the filter device when subjected to a sterilization process, and a closed configuration, where the tubing arrangement is sealed from a surrounding environment. The breathable microbial barrier may at least partially enclose the port of the tubing arrangement.

In some instances, the breathable microbial barrier may be incorporated into a header of a header bag that defines an interior volume. The filter device and the tubing arrangement may be disposed within the interior volume of the header bag. In some other instances, the breathable microbial barrier may include an elasticized band that enables the breathable microbial barrier to be disposed over the port of the tubing arrangement. In some further instances, the tubing arrangement further include a tee fitting, a first tubing, a second tubing, and a third tubing. The tee fitting may have a first end, a second end, and a third end. The first tubing may connect the first end of the tee fitting to the at least one opening of the filter device. The second tubing may connect the second end of the tee fitting to the aseptic connector. The third tubing may by coupled to the third end of the tee fitting. In some additional instances, the sealable component may be a spring actuated valve component that includes a male portion and a female portion. The female portion may be removably coupled to the male portion such that, when the female portion is coupled to the male portion, the sealable component may be in the open configuration, and when the female portion is uncoupled from the male portion, the sealable component may be in the closed configuration.

In yet another embodiment, a method for sterilizing a filter device may include equipping at least one opening of the filter device with a tubing arrangement, enclosing at least the port of the tubing arrangement with a breathable microbial barrier, subjecting the filter device to a sterilization process, and converting the sealable component of the tubing arrangement from an open configuration to a closed configuration. The tubing arrangement may include an aseptic connector, a sealable component disposed in the tubing arrangement between the at least one opening of the filter device and the aseptic connector, and a port.

In some instances, the sterilization process may be autoclaving. In some other instances, the sterilization process may be a gas- or vapor-based sterilization process. In some additional instances, the breathable microbial barrier may include an elasticized band that enables the breathable microbial barrier to be disposed over the port of the tubing arrangement. In some further instances, the breathable microbial barrier may be a portion of a container that defines an interior volume. The container may be a header bag and the breathable microbial barrier may be formed as a header of the header bag. In some even further instances, step of enclosing at least the port of the tubing arrangement within the breathable microbial barrier may further include sealing the filter device and the tubing arrangement within the interior volume of the container. In yet some further instances, the sealable component of the tubing arrangement may be converted to the closed configuration while the filter device is sealed within the container.

### BRIEF DESCRIPTION OF THE DRAWINGS

The apparatuses, systems, devices, modules, components, valves, etc., presented herein may be better understood with reference to the following drawings and description. It should be understood that some elements in the figures may not necessarily be to scale and that emphasis has been placed upon illustrating the principles disclosed herein. In the figures, like-referenced numerals designate corresponding parts/steps throughout the different views.

FIGS. 1A-1F illustrate various views of a modular depth filter device, comprising an insert plate, connectors, and a process scale pod (PSP), according to some embodiments of the disclosure.

FIG. 2 illustrates a top view of a header bag used to sterilize the filter device illustrated in FIGS. 1A-1F in accordance with the embodiments of the present disclosure.

FIG. 3 illustrates a side elevational view of a first side of a modular depth filter device equipped with a tubing arrangement having spring actuated valve components with removable portions in accordance with an embodiment of the present disclosure.

FIG. 4 illustrates a side elevational view of a second side of the modular depth filter device illustrated in FIG. 3.

FIG. 5 illustrates an isolated perspective view of one of the tubing arrangements of the modular depth filter device illustrated in FIG. 3, where the tubing arrangement is equipped with the spring actuated valve component illustrated in FIG. 3 and in accordance with the present disclosure.

FIG. 6 illustrates a top view of the header bag illustrated in FIG. 2 in which the modular depth filter device illustrated in FIG. 3 is disposed, and where the spring actuated valve components are in the open configuration.

FIG. 7 illustrates a perspective view of the one of the tubing arrangements of the modular depth filter device illustrated in FIG. 3, where the tubing arrangement is in the open configuration.

FIG. 8 illustrates a schematic illustration of one of the tubing arrangements of the modular depth filter device illustrated in FIG. 3, where the tubing arrangement is in the open configuration.

FIG. 9 illustrates a top view of the header bag illustrated in FIG. 2 in which the modular depth filter device illustrated in FIG. 3 is disposed, and where the spring actuated valve components are in the closed configuration.

FIG. 10 illustrates a perspective view of the one of the tubing arrangements of the modular depth filter device illustrated in FIG. 3, where the tubing arrangement is in the closed configuration.

FIG. 11 illustrates a schematic illustration of one of the tubing arrangements of the modular depth filter device illustrated in FIG. 3, where the tubing arrangement is in the closed configuration.

FIG. 12 illustrates a perspective view of a cap for the male portion of the spring actuated valve component of the tubing arrangement illustrated in FIG. 5.

FIG. 13 illustrates a side elevational view of the first side of a modular depth filter device illustrated in FIG. 3 that is equipped with the tubing arrangement illustrated in FIG. 5 that is in the closed configuration and equipped with the cap illustrated in FIG. 12.

FIG. 14 illustrates is a side elevational view of a first side of a modular depth filter device equipped with pinch clamp components in accordance with an embodiment of the present disclosure.

FIG. 15 illustrates a perspective view of a tubing arrangement of the modular depth filter device illustrated in FIG. 14, where the tubing arrangement is equipped with a pinch clamp component.

FIG. 16 illustrates a perspective view of a pinch clamp component as illustrated in FIG. 15.

FIG. 17 illustrates a schematic illustration of one of the tubing arrangements of the modular depth filter device illustrated in FIG. 14, where the tubing arrangement is in the open configuration.

FIG. 18 illustrates a schematic illustration of one of the tubing arrangements of the modular depth filter device illustrated in FIG. 14, where the tubing arrangement is in the closed configuration.

FIGS. 19-21 illustrate perspective views of a rotatable sleeve valve component, where the rotatable sleeve valve component may be equipped into a tubing arrangement of a modular depth filter in accordance with an embodiment of the present disclosure.

FIG. 22 illustrates a schematic illustration of a tubing arrangement of a modular depth filter device equipped with the rotatable sleeve valve component illustrated in FIGS. 19-21, where the tubing arrangement is in the open configuration.

FIG. 23 illustrates a schematic illustration of the tubing arrangement illustrated in FIG. 22, where the tubing arrangement is in the closed configuration.

FIG. 24 illustrates a top view of a 4-way stopcock component, where the stopcock may be equipped onto a tubing arrangement of a modular depth filter in accordance with an embodiment of the present disclosure.

FIG. 25 illustrates a schematic illustration of a tubing arrangement of a modular depth filter device equipped with the 4-way stopcock component illustrated in FIG. 24, where the tubing arrangement is in the open configuration.

FIG. 26 illustrates a schematic illustration of the tubing arrangement illustrated in FIG. 25, where the tubing arrangement is in the closed configuration.

FIG. 27 illustrates a tubing arrangement of a modular depth filter device equipped with a single-actuation stopcock component in accordance with the present invention, the single-actuation stopcock component being in the open configuration.

FIG. 28 illustrates the tubing arrangement illustrated in FIG. 27, where the single-actuation stopcock component is in the closed configuration.

FIG. 29 illustrates a schematic illustration of another embodiment of the single-actuation stopcock component illustrated in FIG. 27.

FIGS. 30A and 30B illustrates schematic illustrations of additional embodiments of the single-actuation stopcock component illustrated in FIG. 27.

FIG. 31 illustrates a schematic illustration of a tubing arrangement of a modular depth filter device equipped with a magnetic valve component in accordance with the present disclosure, where the tubing arrangement is in the open configuration.

FIG. 32 illustrates a schematic illustration of the tubing arrangement illustrated in FIG. 31, where the tubing arrangement is in the closed configuration.

FIG. 33 illustrates a flowchart of the steps for sterilizing a filter device with any of the tubing arrangements illustrated in FIGS. 3-29, 30A, 30B, 31, and 32 and for maintaining the sterility of the filter device.

FIG. 34 illustrates a side elevational view of a modular depth filter device equipped with spring actuated shut-off valve components having removable portions in accordance with an embodiment of the present disclosure, where the ends of the removable portions are equipped with a breathable microbial barrier.

FIG. 35 illustrates a side view of one of the tubing arrangements of the modular depth filter device illustrated in FIG. 34, where the tubing arrangement is equipped with a spring actuated shut-off valve component and is in the open configuration.

FIG. 36 illustrates a side view of the tubing arrangement illustrated in FIG. 36, where the tubing arrangement is being transitioned from the open configuration to the closed configuration via the removal of one of the removable portions of the spring actuated shut-off valve component.

FIG. 37 illustrates a side elevational view of the modular depth filter device illustrated in FIG. 34, where the removable portions of the spring-actuated shut-off valve components are removed and the tubing arrangements are in the closed configuration.

FIG. 38 illustrates a side view of the tubing arrangement illustrated in FIG. 36, where the tubing arrangement is in the closed configuration.

FIG. 39 illustrates a flowchart of the steps for sterilizing a filter device with the tubing arrangement illustrated in FIGS. 34-39 and for maintaining the sterility of the filter device.

FIG. 40 illustrates a side view of a tubing arrangement of a modular depth filter device, where the tubing arrangement may be utilized for in-process leakage integrity testing, the tubing arrangement being equipped with a spring actuated shut-off valve component and being in the open configuration.

FIG. 41 illustrates the tubing arrangement illustrated in FIG. 40, where the tubing arrangement is in the closed configuration and after leakage testing is complete.

FIG. 42 illustrates a flowchart of the steps for testing a filter device with the tubing arrangement illustrated in FIGS. 34-39 for leaks.

### DETAILED DESCRIPTION

Aspects of the disclosure are disclosed in the description herein. Alternate embodiments of the present disclosure and their equivalents may be devised without parting from the spirit or scope of the present disclosure. It should be noted that any discussion herein regarding “one embodiment,” “an embodiment,” “an exemplary embodiment,” and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of a given embodiment may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

Although specific terms are used in the following description for the sake of clarity, these terms are intended to refer only to the particular structure of the embodiments selected for illustration in the drawings. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2% to 10%” is inclusive of the endpoints, 2% and 10%, and all the intermediate values).

As used herein, approximating language may be applied to modify any quantitative representation that may vary without resulting in a change in the basic function to which it is related. Accordingly, a value modified by a term or terms, such as “about,” “approximately,” and “substantially,” may not be limited to the precise value specified, in some cases. The modifiers should also be considered as disclosing the range defined by the absolute values of the two endpoints. For example, the expression “from about 2 to about 4” also discloses the range “from 2 to 4.”

It should be noted that some terms used herein are relative terms. For example, the terms “upper” and “lower” are relative to each other in location, i.e., an upper component is located at a higher elevation than a lower component and should not be construed as requiring a particular orientation or location of the structure. As a further example, the terms “interior,” “exterior,” “inward,” and “outward” are relative to a center, and should not be construed as requiring a particular orientation or location of the structure.

The terms “top” and “bottom” are relative to an absolute reference, i.e., the surface of the earth. Put another way, a top location is always located at a higher elevation than a bottom location, toward the surface of the earth.

The terms “sterilization,” “sterilized,” and “sterile,” typically refer to sterilization processes or conditions that result in a sterility assurance level (SAL) of 10⁻⁶, which represents a 1 in 1,000,000 chance of a non-sterile unit. For the purposes of the present disclosure, these terms shall also comprise sub-sterilization processes and conditions where 10⁻⁶ SAL sterility is not achieved, which may sometimes be denoted using higher SALs (e.g., 10⁻⁵, 10⁻⁴, 10⁻³, etc.) and/or other terms like “bioburden reduction,” “bioburden-reduced,” “sanitization,” and/or “sanitary.”

“TFF assembly,” “TFF system,” and “TFF apparatus” are used interchangeably herein to refer to a tangential flow filtration system that is configured for operation in a recirculation mode where at least a portion of the retentate is returned to the system as feed.

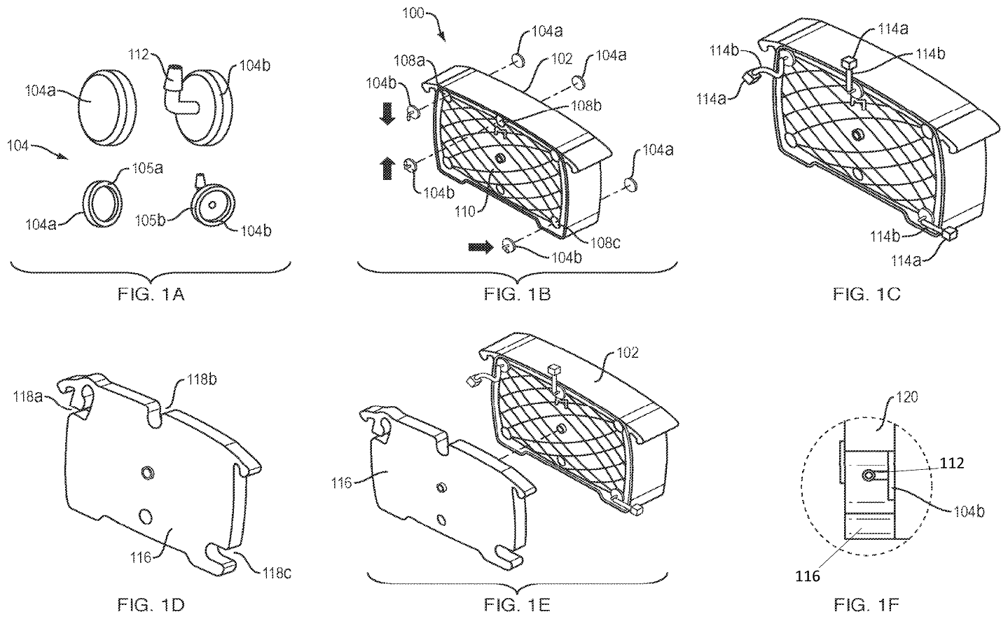

Turning now to FIGS. 1A-1C, depicted are views of a modular depth filter device (pod) 100, comprising an insert plate 116, connectors 114a, and a process scale pod 102, according to embodiments of the disclosure. The modular depth filter device 100 shown is a process scale pod 102 having one or more add-on components 104 (e.g., as shown in FIG. 1A). The modular depth filter device 100 is closed from the environment during the entire use cycle of the device and enables the aseptic connection and disconnection of this device for closed processing applications. FIG. 1A shows a blind end component 104a and a 90° elbow hose barb connection component 104b. In some embodiments, there is a rim 105a, 105b for each component (rear side shown in FIG. 1A). In FIG. 1B, the blind end component(s) 104a are attached to the pod 102, which has 6 port openings 108 (such as 108a, 108b, 108c) in total. More specifically, the pod 102 may have three port openings on a rear face 111 (not shown) in fluid communication and opposite to three port openings 108a, 108b, 108c on a front face 110. Three hose barb fitting or connector(s) 112, at a terminal end of hose barb connection components 104b, are attached to the three front port openings 108a, 108, 108b, while the blind end components 104a are attached to the three rear openings by any suitable plastic joining method. While the pod 102 is shown in this configuration, the hose barb connection component 104b and the blind end components 104a may be attached to either face 110, 111 of the pod 102. For example, instead of the three hose barb fittings 112 being disposed on the front face 110 and the three blind end components 104a being disposed on the rear face 111, two hose barb components 104b and one blind end components 104a may be disposed on the front face 110 while one hose barb component 104b and two blind end components 104a are disposed on the rear face 111. Having blind components 104a disposed all on one side/face 110, 111 can make that particular side/face 110, 111 appear flat. For example, plastic joining methods include, but are not limited to, solvent bonding, vibration welding, laser welding, and induction welding techniques. A 90° elbow hose barb connection component 104b may also be attached by spin welding due to the circular geometry of the interface. Tubing 114b (e.g., silicone or C-Flex® (Saint Gobain)) may be installed onto each hose barb fitting 112 and a sterile-to-sterile or aseptic connector 114a (e.g., AseptiQuik® G (Colder Products Corp.), ReadyMate™ disposable aseptic connectors (Cytiva), LYNX® S2S Connector (manufactured by EMD Millipore Corporation), KLEENPAK® Presto sterile connectors (Pall Corp.), and PURE-FITR SC (Saint Gobain)) is attached to the end of the tubing 114b as illustrated in FIG. 1C. The aseptic connectors 114a illustrated in FIGS. 1C and 1E are shown as cubes for illustrative purposes only. The type of aseptic connector component can be chosen depending on application, tubing size, sterilization method compatibility, etc. Alternatively, a blind end component 104a and a 90° elbow hose barb connection component 104b can be integrated into each end plate during the injection molding of those parts. In addition, instead of the hose barb fittings 112, tri-clover or tri-clamp sanitary fittings may be used. Furthermore, instead of the elbow in the hose barb fittings 112 in the hose barb connection component 104b being 90°, other angles such as 135° or 180° (or straight hose barb) may be utilized. The modular depth filter device 100 with tubing 114b and aseptic connectors 114a are considered as one module, which is to be packaged and sterilized (or bioburden-reduced) before use (e.g., gamma irradiation, X-ray irradiation, electron-beam/beta irradiation, ethylene oxide, vaporized hydrogen peroxide, nitrogen dioxide, vaporized peracetic acid, or autoclave) as explained in further detail below.

As shown in FIG. 1C, the orientation of the 90° elbow of the hose barb fitting 112 of each hose barb connection component 104b can vary. For example, the hose barb fitting 112 may face down with respect to any port opening 108a, 108b, 108c, which may be an inlet, e.g., 108a, upwards for a vent port, e.g., 108b, and right facing for an outlet port, e.g., 108c. Other orientations of the hose barb connectors are possible. Typically, the vent port 108b comprises a hose barb fitting 112 that is facing upwards to facilitate air removal. FIG. 1D shows an insert plate 116 and the location and geometry of each slot 118a, 118b, 118c in the insert plate correspond to each of the hose barb fittings 112 and the hose barb connection components 104b. The insert plate 116 as shown in FIG. 1D has one slot 118a, 118b, 118c at each side/edge (i.e., left, top, and right), which helps an end user easily identify from which port each tubing 114b and its associated aseptic connector 114a originates. The insert plate 116 may be made of plastic, metal, ceramics or combinations thereof. The insert plate 116 is a separate part, which is single-use or reusable, and is not bonded to the modular depth filter device 100 as shown in FIG. 1E. The insert plate 116 has a suitable thickness to contain and protect the hose barb fittings 112 and their associated tubing 114b. That is, when the insert plate 116 is in the assembled condition attached to a face 110, 111 of the pod 102, the location and configuration of each of the slots 118a, 118b and 118c in the insert plate 116 causes the hose barb fittings 112 and associated tubing 114b to be recessed within the thickness of the insert plate 116. FIG. 1F illustrates a hose barb connection component 104b and associated hose barb fitting 112 being disposed within an inset 120 at the outlet location when the insert plate 116 is placed in contact with an end plate of an adjacent modular depth filter device 100. Depending on the size of hose barb connection component 104b and/or the weight of each insert plate 116, additional insert plates 116 (not shown) can be placed between two filter devices 100 to create room for a hose barb connection component 104b, the associated hose barb fitting 112, and the associated tubing 114b. The purpose of the insert plate 116 is to contain and protect the hose barb connection component 104b, the hose barb fittings 112, and the tubing 114b when multiple modular depth filter devices 100 are stacked together for full scale operation. Without the insert plate 116, the hose barb connection component 104b, the hose barb fittings 112, and the attached tubing 114b can become damaged, pinched, or inoperable as intended when the devices 100 are compressed together in a holder hardware during operation using a press, for example, a hydraulic pump.

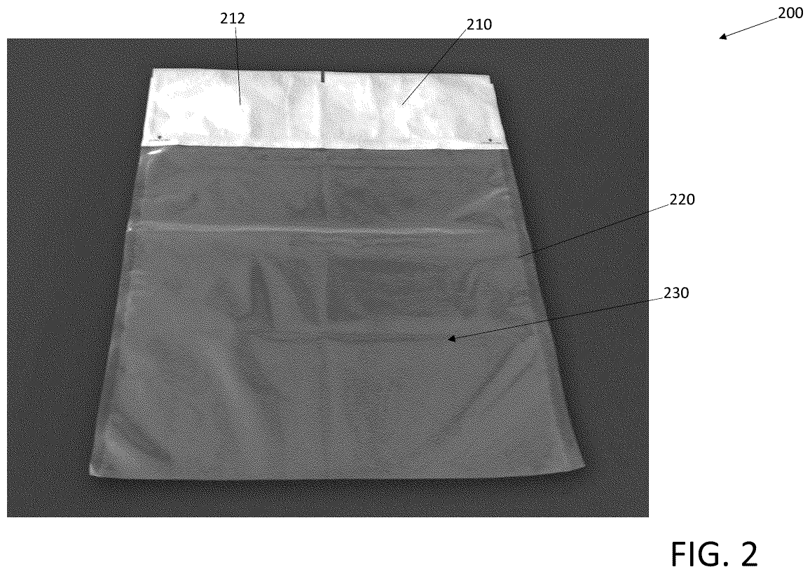

Turning to FIG. 2, illustrated is a header bag 200 that may be utilized in the sterilization process of a filter device, including, but not limited to, the modular depth filter device 100, in accordance with the present invention. The header bag may contain a first portion 210 and a second portion 220 that collectively define an interior volume 230. The second portion 220 may be constructed or formed from a peelable polypropylene laminate, where the second portion 220 is impermeable. The first portion 210 may include a header 212 that is constructed from high-density polyethylene fibers that are either interwoven or flashspun to create a material that is vapor permeable (i.e., water vapor) while also being capable of withstanding gamma irradiation or ethylene oxide gas used to sterilize equipment. The vapor permeability of the header 212 of the header bag 200 allows objects, after being placed and sealed within the interior volume 230 of the header bag 200, to be subjected to a sterilization process (e.g., autoclaving, ethylene oxide, chlorine dioxide, ozone, supercritical carbon dioxide, and vaporized hydrogen peroxide, etc.) while being disposed within the interior volume 230 of the header bag 200. The polypropylene laminate of the second portion 220 and the high-density polyethylene of the header 212 enable the contents of a header bag 200 to remain sterile after being subjected to the sterilization process within the header bag 200 and without any further manipulation or reconfiguration of the header bag 200.

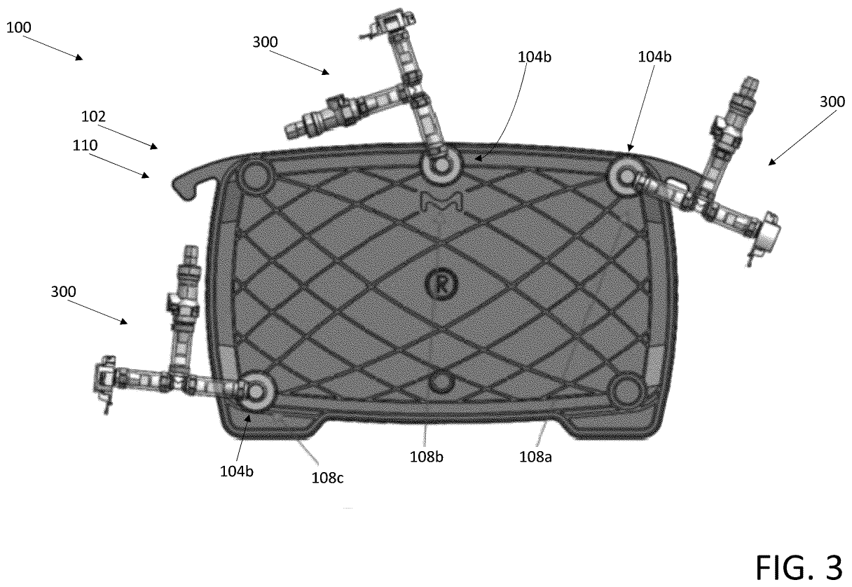

Turning to FIGS. 3-11, and with continued reference to FIGS. 1A-IF and 2, illustrated is a modular depth filter device 100 equipped with a first embodiment of a tubing arrangement 300 that facilitates sterilization of the modular depth filter device 100 with the header bag 200. While a modular depth filter device 100 is illustrated, the modular depth filter device 100 is intended as an example only. The tubing arrangement 300 illustrated in FIGS. 3-11 may be utilized with any other type of filter device to facilitate sterilization of that particular filter device.

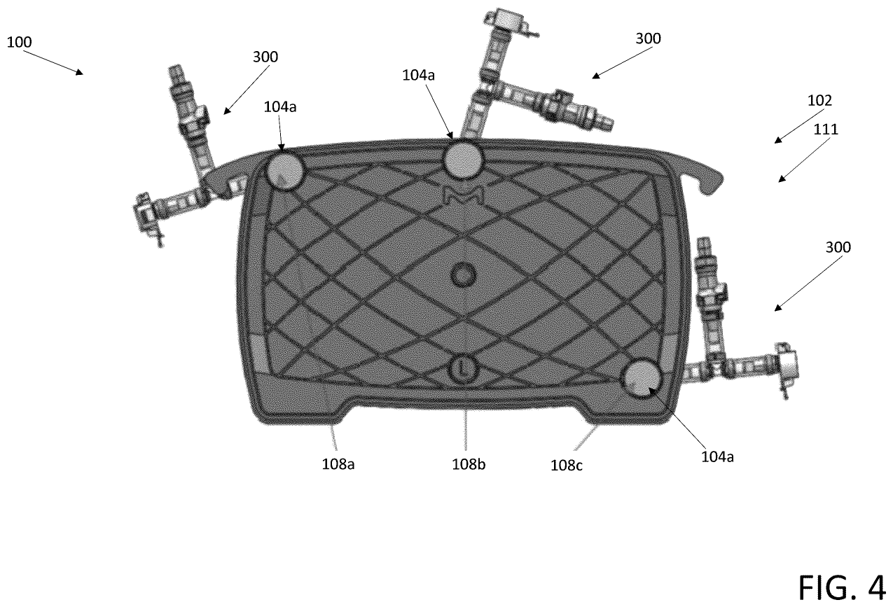

As best illustrated in FIG. 3, hose barb connection components 104b are installed and disposed on the front face 110 of the pod 102 of the modular depth filter device 100 at the port openings 108a, 108b, 108c, where tubing arrangements 300 are coupled to the hose barb fittings 112 of the hose barb connection components 104b. As best illustrated in FIG. 4, blind end components 104a are installed and disposed on the rear face 111 of the pod 102 of the modular depth filter device 100 at the port openings 108d, 108e, 108f, which are in fluid communication and opposite to the port openings 108a, 108b, 108c, respectively, disposed on the front face 110. As previously explained, while the pod 102 is shown in this configuration, the hose barb connection components 104b and the blind end components 104a may be attached to either face 110, 111 of the pod 102.

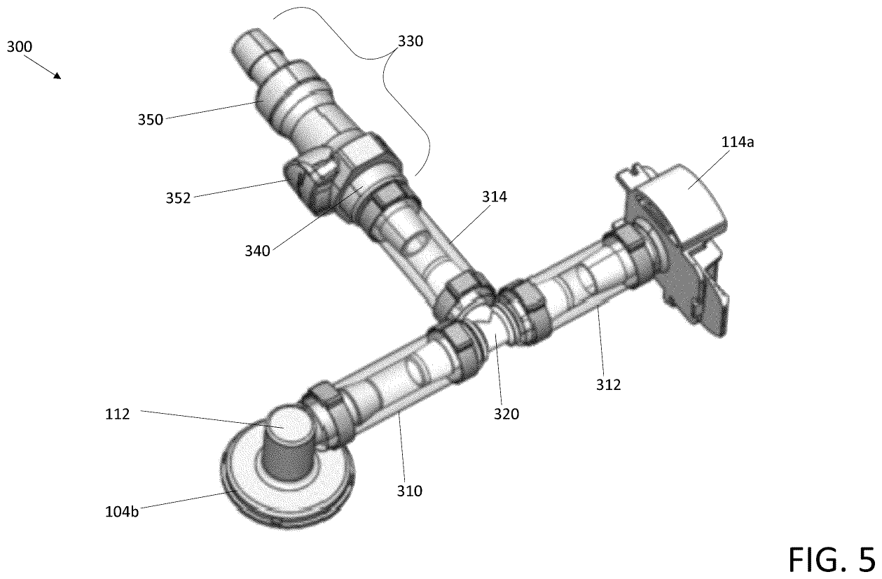

FIG. 5 depicts the tubing arrangement 300 of the filter device 100 that, as explained in further detail below, enables the filter device 100 to be subjected to a sterilization process while maintaining the sterility of the filter device 100 after completion of the sterilization process. While FIG. 5 only depicts a single tubing arrangement 300, the tubing arrangement 300 may represent any of the tubing arrangements 300 illustrated in FIGS. 3 and 4. The tubing arrangement 300 may include a series of tubing 310, 312, 314 that connected to one another via a tee fitting 320. As further illustrated, tubing 310 may be connected between the hose barb fitting 112 of the hose barb connection component 104b and the tee fitting 320. Tubing 312 may be connected between an aseptic connector 114a and the tee fitting 320. Furthermore, tubing 314 may be connected between a valve component 330. The valve component 330 may be a spring actuated valve component having a male portion 340 and a female portion 350 that are capable of being disconnected from one another. In some embodiments, the valve component 330 may be male and female HFC39 disconnect components. In FIG. 5, the female portion 350 is shown coupled to the male portion 340, where the male portion 340 is inserted into the female portion 350. The female portion 350 may further include an actuator 352 that, when actuated, may facilitate the disconnection or uncoupling of the female portion 350 from the male portion 340.

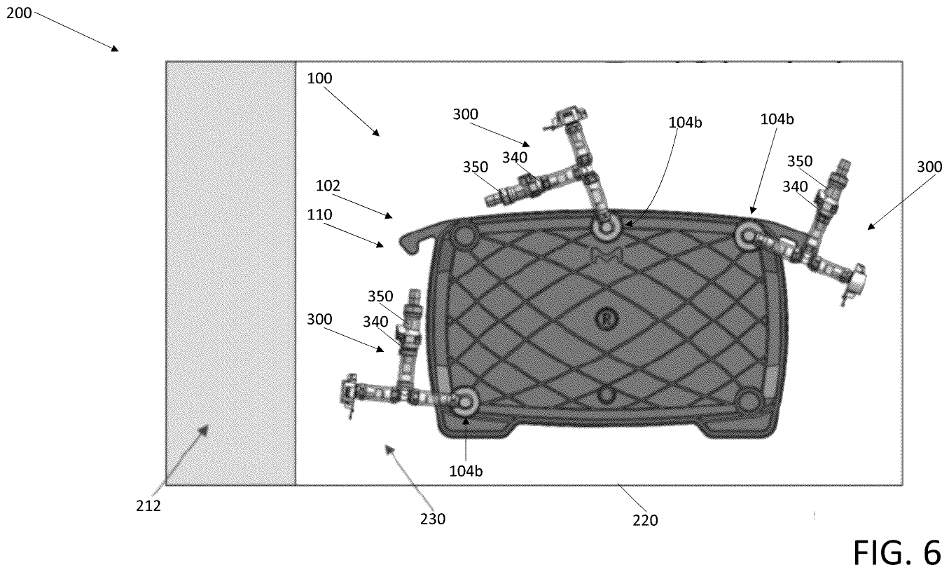

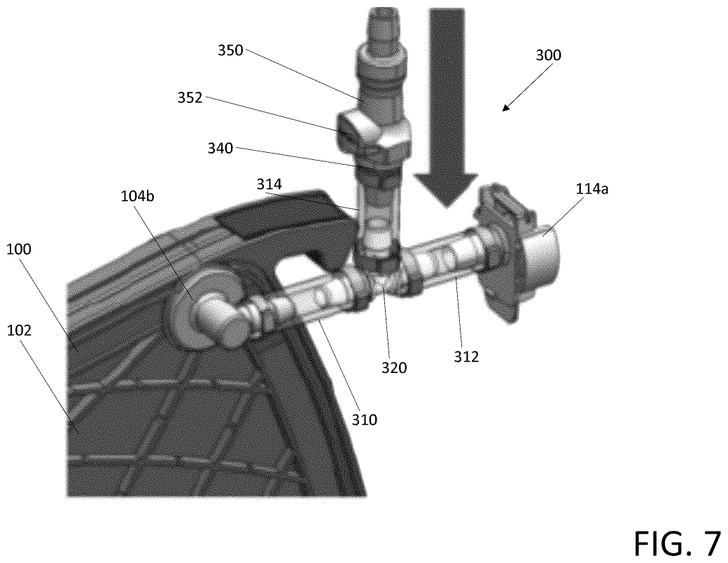

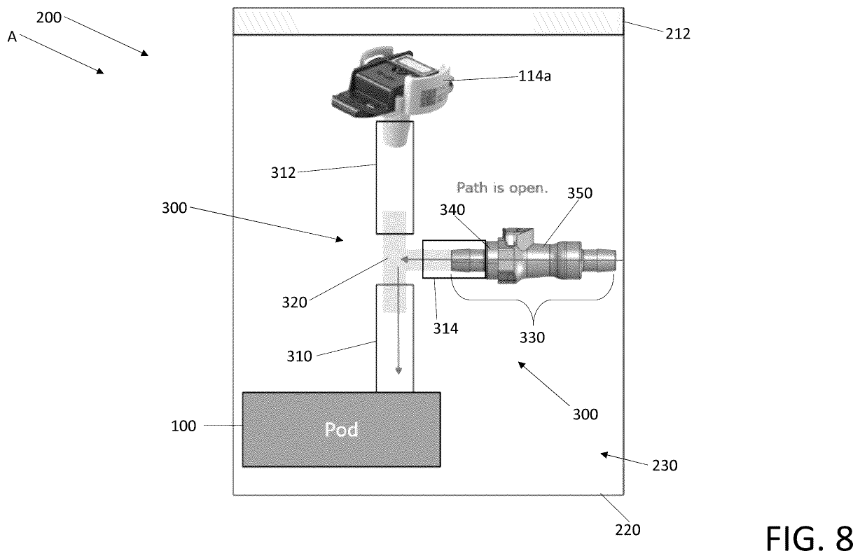

As best illustrated in FIGS. 6-8, the valve component 330 is in the open state A. When the female portion 350 of the valve component 330 is coupled to the male portion 340, the valve component 330 is in the open state A. In the open state A, a fluid (e.g., air, vapor, etc.) may flow through the valve component 330 and into the filter device 100 via tubing 314, the tee fitting 320, tubing 310, and the hose barb connector component 104b. This flowpath is best shown in the schematic illustration of FIG. 8. With the female portion 350 of the valve component 330 of each tubing arrangement 300 coupled to the respective male portion 340, each tubing arrangement 300 of the filter device 100 provides a fluid pathway into the filter device 100. When the tubing arrangements 300 are in the open states A, the filter device 100 may be placed into the interior volume 230 of a header bag 200 like that shown in FIG. 6. Once in the header bag 200, or immediately prior to inserting the filter device 100 into the header bag 200, a user may verify that the female portions 350 of the vale components 330 of the tubing arrangements 300 are fully coupled to the respective male portions 340 like that shown in FIG. 7 (i.e., the male portions 340 are fully inserted into the female portion 350).

The header bag 200 encasing the modular depth filter device 100 may then be subjected to a sterilization process (e.g., autoclaving and other gas- or vapor-based sterilization methods, such as, but not limited to, ethylene oxide, chlorine dioxide, ozone, supercritical carbon dioxide, and vaporized hydrogen peroxide) where a sterilizing vapor may flow into the interior volume 230 of the header bag 200 through the header 212, and then into the filter device 100 via the tubing arrangements 300. As previously explained and best shown in the schematic illustration of FIG. 8, when the female portion 350 is inserted into and connected to the male portion 340, the tubing arrangement 300 provides an open pathway for the sterilizing vapor to flow into the filter device 100. More specifically, the sterilization vapor may flow into the tubing arrangement 300 via the valve component 330, through the tubing 314, through the tee fitting 320, and through the tubing 310 into the filter device 100. While FIG. 8 illustrates a single schematic illustration of a tubing arrangement 300, this schematic illustration is for illustrative purposes only, and the illustrated flow path is represented of a flow path of each of the tubing arrangements 300 of the filter device 100 when in the open state A.

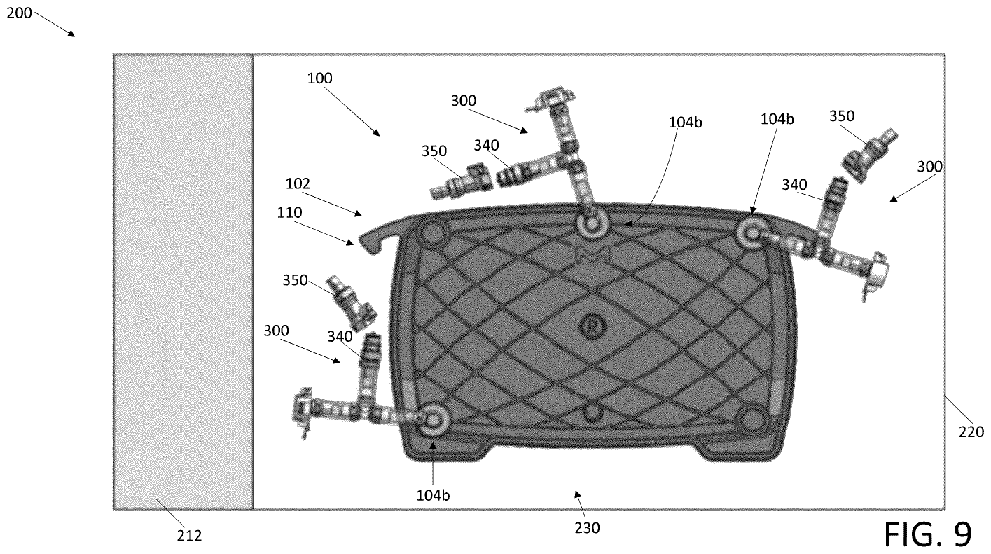

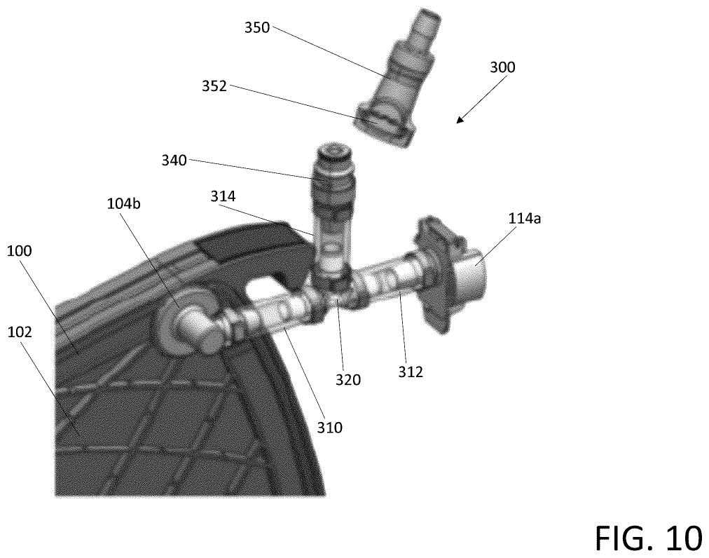

As best illustrated in FIGS. 9-11, the valve component 330 is in the closed state B due to the female portion 350 of the valve component 330 being uncoupled or disconnected from the male portion 340. When in the closed state B, a fluid (e.g., air, vapor, etc.) is unable to flow through the valve component 330 and into the filter device 100. Thus, when the female portion 350 of the valve component 330 of each tubing arrangement 300 is uncoupled/disconnected from the respective male portion 340, the filter device 100 no longer contains a fluid pathway into the interior of the filter device 100. The male portion 340 of the valve component 330 may be equipped with a spring actuated valve (not shown) that closes when the female portion 350 is disconnected from the male portion 340.

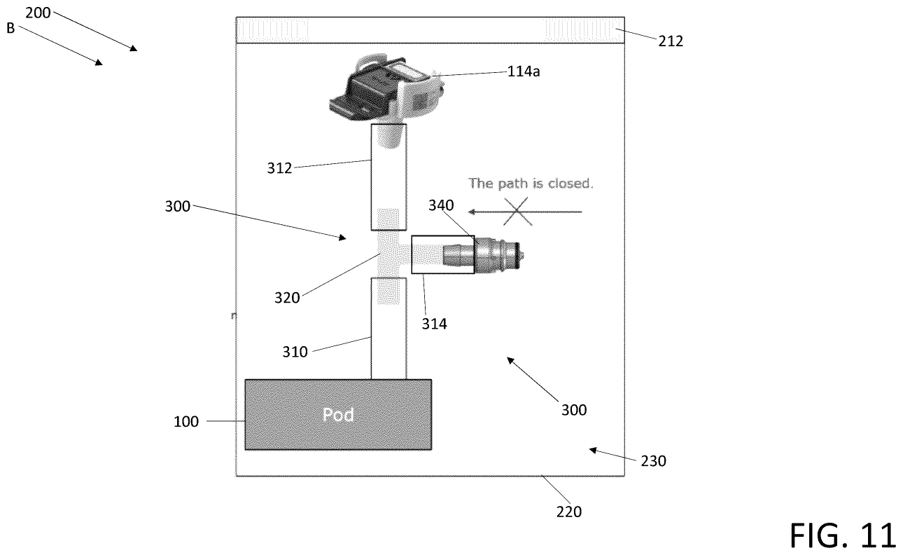

As best illustrated in FIG. 9, after the header bag 200 and the filter device 100 have been subjected to a sterilization process (e.g., autoclaving and/or other gas- or vapor-based sterilization methods, such as, but not limited to, ethylene oxide, chlorine dioxide, ozone, supercritical carbon dioxide, and vaporized hydrogen peroxide), and prior to the filter device 100 being removed from the header bag 200, the female portion 350 of the valve component 330 of each of the tubing arrangements 300 is removed from its respective male portion 340 to convert the tubing arrangements 300 from the open state A to the closed state B. FIG. 10 further illustrates the female portion 350 being removed from the male portion 340 of a valve component 330 prior to the filter device 100 being removed from the header bag 200. More specifically, the actuator 352 of the female portion 350 may be actuated (e.g., depressed, etc.) in order to facilitate the removal or disconnection of the female portion 350 from the male portion 340. As previously explained, the male portion 340 of the valve component 330 may be equipped with a spring actuated valve (not shown) that closes when the female portion 350 is disconnected from the male portion 340. This is further illustrated in the schematic illustration of FIG. 11, which depicts that when the female portion 350 is disconnected from the male portion 340, the pathway of the tubing arrangement 300 into the filter device 100 is blocked or closed off. Thus, a sterilizing vapor is unable to flow into the filter device 100. While FIG. 11 illustrates a single schematic illustration of a tubing arrangement 300, this schematic illustration is for illustrative purposes only, and the illustrated blocked flow path is represented of closed off flow path of each of the tubing arrangements 300 of the filter device 100 when in the closed state B.

Once the filter device 100 has been sterilized and the tubing arrangements 300 are converted into the closed state B, the filter device 100 may be removed from the interior volume 230 of a header bag 200. Because the male portion 340 of each of the valve components 330 contains a spring actuated valve that closes when the respective female portion 350 is disconnected, the sterility of the interior of filter device 100 is maintained once the filter device 100 is removed from the header bag 200 and the tubing arrangements 300 are retained in the closed state B.

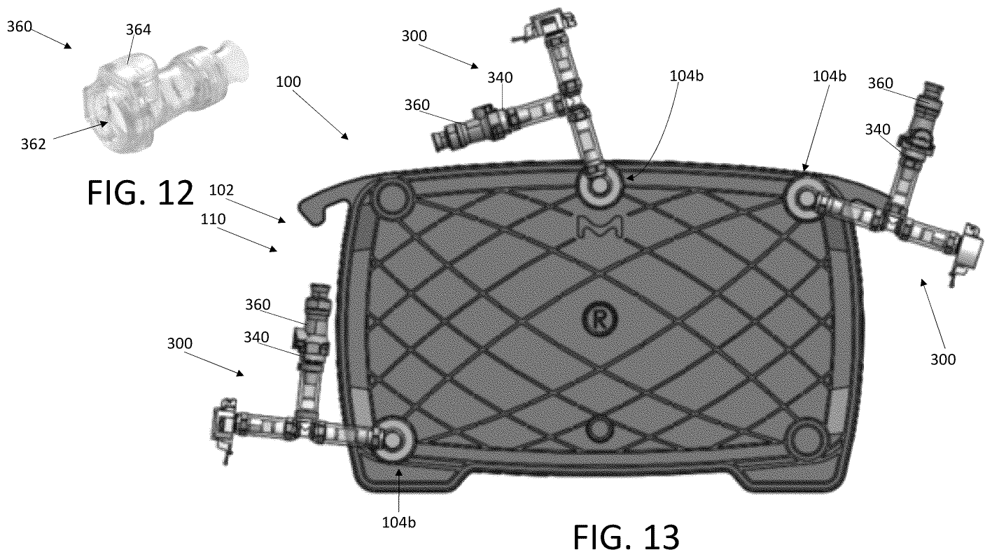

In some embodiments, once the filter device 100 has been sterilized and removed from the header bag 200, a cap 360, as illustrated in FIGS. 12 and 13, is fitted onto the end of the male portion 340 of the valve component 330. As best illustrated in FIG. 12, the cap 360 may include a receptacle 362 at one end and an actuator 364. The receptacle 362 may be configured to receive the end of the male portion 340 to couple the cap 360 to the male portion 340 of the valve component 330. The actuator 364 may function in a similar manner to the actuator 352 of the female portion 350, where actuation (e.g., depression, etc.) of the actuator 364 may serve to uncouple or disconnect the cap 360 from the male portion 340 of the valve component 330. With the filter device 100 removed from the header bag 200, but prior to use of the filter device 100, the cap 360 may be optionally connected to each male portion 340 of the valve component 330 of the tubing arrangement 300 like that illustrated in FIG. 13 to prevent accidental actuation of the spring valve of the male portion 340. The cap 360 may not impact the sterility of the male portion 340 of the valve component 330 and the remaining portions of the tubing arrangement 300 because the cap 360 may not interact with the spring actuated valve of the male portion 340 of the valve component 330. Thus, placing the cap 360 on the male portion 340 of the valve component 330 of each of the tubing arrangements 300 will not adversely affect the sterility behind the male portion 340 of the valve component 330 of the tubing arrangement 300.

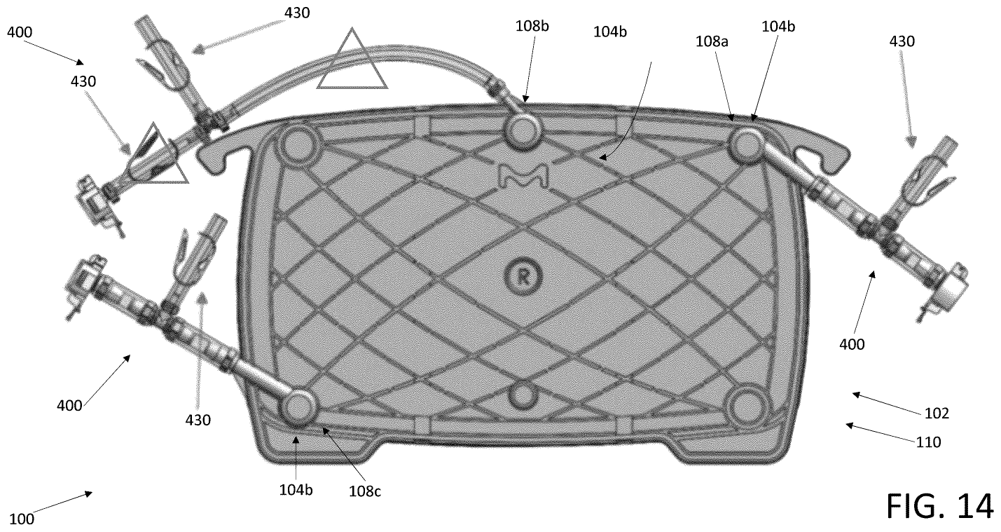

Turning to FIGS. 14-18, and with continued reference to FIGS. 1A-IF and 2-13, illustrated is a second embodiment of a tubing arrangement 400 that facilitates sterilization of a modular depth filter device 100 with the header bag 200. As best illustrated in FIG. 14, a modular depth filter device 100 may be equipped with hose barb connection components 104b are installed and disposed on the front face 110 of the pod 102 of the modular depth filter device 100 at the port openings 108a, 108b, 108c, where tubing arrangements 400 are coupled to the hose barb connection components 104b. While not illustrated, blind end components 104a may be installed and disposed on the rear face 111 of the pod 102 of the modular depth filter device 100 at the port openings 108d, 108e, 108f (not shown), which are in fluid communication and opposite to the port openings 108a, 108b, 108c, respectively, disposed on the front face 110. As previously explained, while the pod 102 is shown in this configuration, the hose barb connection components 104b and the blind end components 104a may be attached to either face 110, 111 of the pod 102.

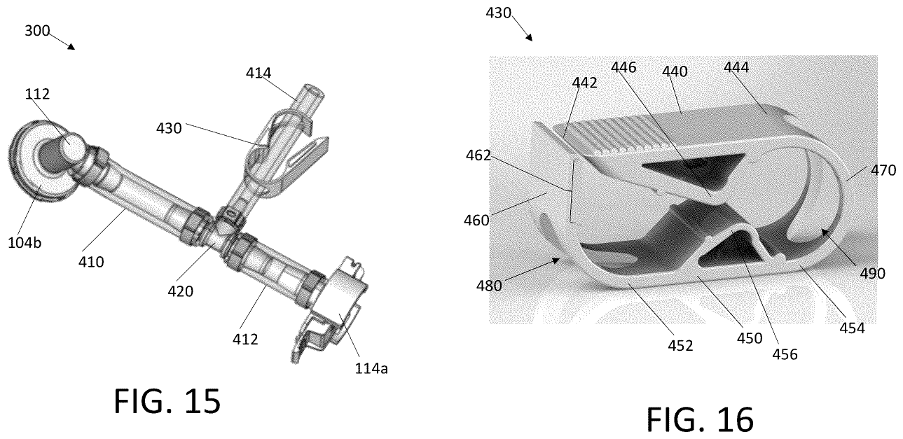

FIGS. 14 and 15 depict a second embodiment of the tubing arrangement 400 of the filter device 100 that, as explained in further detail below, enables the filter device 100 to be subjected to a sterilization process while maintaining the internal sterility of the filter device 100 after completion of the sterilization process. While the tubing arrangement 400 is depicted on a modular depth filter device 100, the modular depth filter device 100 is intended as an example only. The tubing arrangement 400 illustrated in FIGS. 14-18 may be utilized with any other type of filter device to facilitate sterilization of that particular filter device. Moreover, while FIG. 15 only depicts a single tubing arrangement 400, the tubing arrangement 400 may represent any of the tubing arrangements 400 illustrated in FIG. 14. The tubing arrangement 400 may include a series of tubing 410, 412, 414 that connect to one another via a tee fitting 420. As further illustrated, tubing 410 may be connected between the hose barb connection component 104b and the tee fitting 420. Tubing 412 may be connected between an aseptic connector 114a and the tee fitting 420. Furthermore, tubing 414 may extend from the tee fitting 420 and provide an inlet/outlet to the tubing arrangement 400. A pinch clamp 430 may be disposed on the tubing 414, where the pinch clamp 430 may be configured to change the state of the tubing arrangement 400 between the open state C and the closed state D.

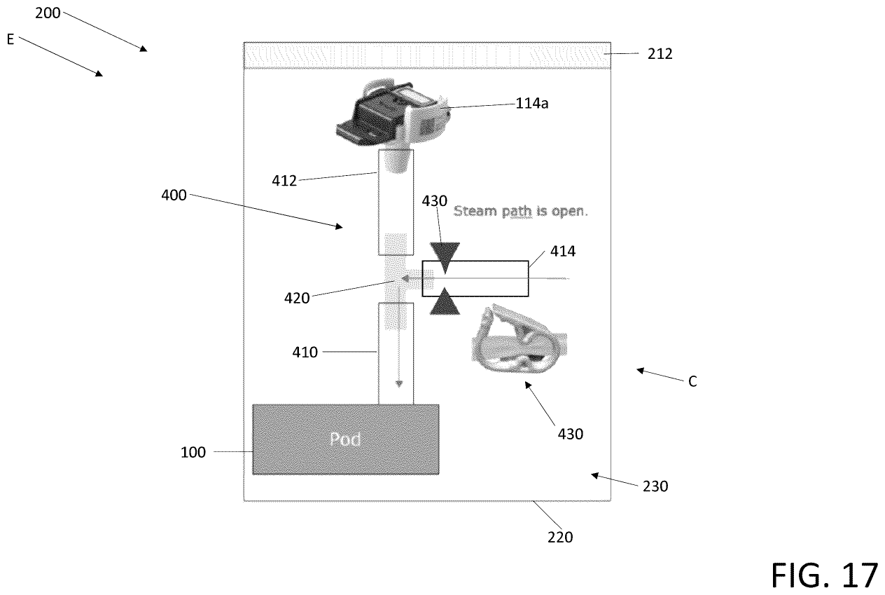

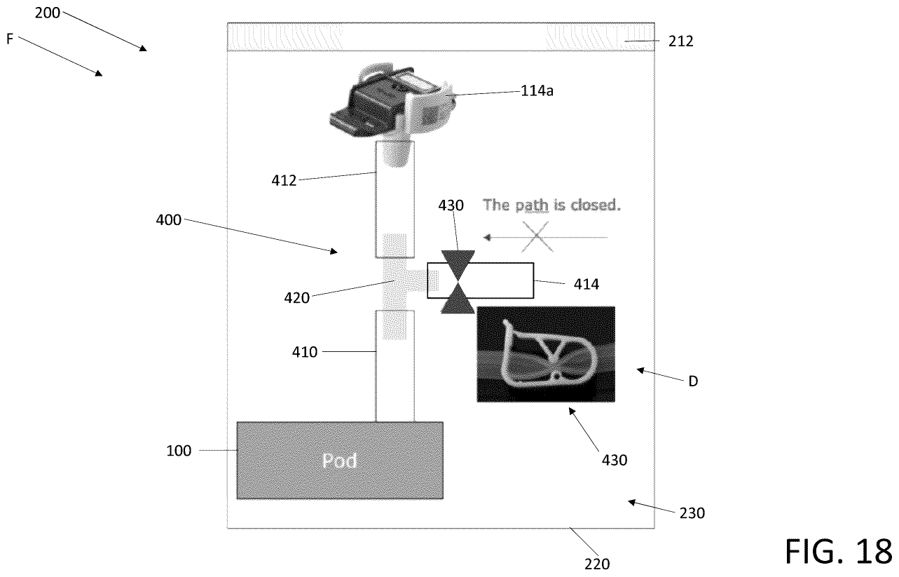

FIG. 16 illustrates an isolated pinch clamp 430 of the tubing arrangement 400. The pinch clamp 430 may include a first clamp arm 440 and a second clamp arm 450 that are oriented substantially parallel to one another. The first clamp arm 440 may include a first end 442 and an opposing second end 444. The first clamp arm 440 may further include a clamp protrusion 446 that is disposed on an inner surface of the first clamp arm 440 between the first and second ends 442, 444. Similarly, the second clamp arm 450 may include a first end 452 and an opposing second end 444. The second clamp arm 450 may also include a clamp protrusion 456 that is disposed on an inner surface of the second clamp arm 450 between the first and second ends 452, 454. As further illustrated in FIG. 16, the clamp protrusion 446 of the first clamp arm 440 extends towards the clamp protrusion 456 of the second clamp arm 450. Extending upwardly from the first end 452 of the second clamp arm 450 is a retaining portion 460. The retaining portion 460 may further include a series of teeth 462 disposed on the inner surface of the retaining portion 460. FIG. 16 further illustrates that a fulcrum portion 470 connects the second end 444 of the first clamp arm 440 to the second end 454 of the second clamp arm 450. The fulcrum portion 470 may be constructed and configured to facilitate movement (e.g., pivot, rotation, etc.) of the first clamp arm 440 with respect to the second clamp arm 450, and vice versa. As further illustrated in FIG. 16, the pinch clamp 430 includes a first opening 480 and a second opening 490, where both openings 480, 490 are configured to receive a portion of the tubing 414. The first opening 480 may be at least partially disposed in the retaining portion 460 and the second clamp arm 450, while the second opening 490 may be disposed at least partially in the fulcrum portion 470 and the second clamp arm 450. As best illustrated in FIG. 15, the tubing 414 may be inserted through the first and second openings 480, 490 in order to dispose the pinch clamp 430 on the tubing 414.

When the tubing 414 is inserted through the openings 480, 490 of the pinch clamp 430, the clamp protrusion 456 of the second clamp arm 450 may be disposed or imparted against the tubing 414. When the pinch clamp 430 is in the unclamped position C, the clamp protrusion 446 of the first clamp arm 440 may be spaced from the tubing 414 or may rest against the tubing 414 without cutting off the fluid pathway through the tubing 414. Furthermore, when the pinch clamp 430 is in the unclamped position C, the first end 442 of the first clamp arm 440 are unengaged with (e.g., spaced from) the teeth 462 of the retaining portion 460 of the pinch clamp 430. Conversely, when the pinch clamp 430 is in the clamped position D, which is best illustrated in FIG. 18, the clamp protrusion 446 of the first clamp arm 440 may be disposed or imparted against the tubing 414 on an opposing side of the tubing 414 from the clamp protrusion 456 of the second clamp arm 450 such that the fluid pathway of the tubing 414 is closed off. In addition, when the pinch clamp 430 is in the clamped position D, the first end 442 of the first clamp arm 440 is engaged with the teeth 462 of the retaining portion 460 to retain the first clamp arm 440 against the tubing 414 and to retain the pinch clamp 430 in the clamped position D.

Returning to FIG. 14, as illustrated, the pinch clamp 430 may also be optionally disposed on the two triangled regions shown in FIG. 14 and is not only limited to being disposed on the tubing 414 of each tubing arrangement 400.

Turning to FIGS. 17 and 18, illustrated are schematic illustrations of a filter device 100 disposed within a header bag 200, where the filter device is equipped with the second embodiment of the tubing arrangements 400 that contain a pinch clamp 430. While FIGS. 17 and 18 each illustrate a schematic illustration of a single tubing arrangement 400, the schematic illustrations are for illustrative purposes only, and the illustrated tubing arrangement 400 may be representative of any of the tubing arrangements 400 of the filter device 100 illustrated in FIG. 14.

FIG. 17 illustrates the filter device 100 equipped with the second embodiment of the tubing arrangements 400 disposed within the header bag 200 and before the filter device 100 has been subjected to a sterilization process (e.g., autoclaving and other gas- or vapor-based sterilization methods, such as, but not limited to, ethylene oxide, chlorine dioxide, ozone, supercritical carbon dioxide, and vaporized hydrogen peroxide). As illustrated in FIG. 17, the pinch clamp 430 is disposed on the tubing of the tubing arrangement 400 and is in the unclamped position C. Thus, the tubing arrangement 400 is in the open state E, where a fluid (e.g., air, vapor, etc.) may flow into and through the tubing arrangement 400 via the tubing 414. Thus, when the header bag 200 containing the filter device 100 equipped with the second embodiment of the tubing arrangement 400 is subjected to a sterilization process, a sterilization vapor may pass through the header 212 of the header bag 200 and into the fluid pathway of the tubing 414 of the tubing arrangement 400. The sterilization vapor may then pass beyond the pinch clamp 430, into the tee fitting 420, through the tubing 410, and into the filter device 100 in order to sterilize the filter device 100.

As best illustrated in FIG. 18, the pinch clamp 430 is in the clamped position D (i.e., the tubing 414 is clamped between the clamp protrusions 446, 456 of the pinch clamp 430) which changes the tubing arrangement 400 from the open state E to the closed state F. When in the closed state F, a fluid (e.g., air, vapor, etc.) is unable to flow through the tubing 414, beyond the pinch clamp 430, and into the filter device 100. Thus, when the pinch clamp 430 is in the clamped position D, the filter device 100 no longer contains a fluid pathway into the interior of the filter device 100.

As best illustrated in FIG. 18, after the header bag 200 and the filter device 100 have been subjected to a sterilization process, and prior to the filter device 100 being removed from the header bag 200, the pinch clamps 430 are converted from the unclamped position C to the clamped position D to convert the tubing arrangements 400 from the open state E to the closed state F. More specifically, a user may compress the pinch clamp 430 such that the first clamp arm 440 is translated toward the second clamp arm 450, which causes the clamp protrusions 446, 456 to translate toward one another and clamp the tubing 414. Once this has been completed for each of the tubing arrangements 400 the interior of the filter device 100 is isolated from its surrounding environment.

Once the filter device 100 has been sterilized and the tubing arrangements 400 are converted into the closed state F, the filter device 100 may be removed from the interior volume 230 of the header bag 200. Because the pinch clamps 430 are in the clamped state D and are cutting off the fluid pathway of the tubing 414, the internal sterility of the filter device 100 is maintained once the filter device 100 is removed from the header bag 200.

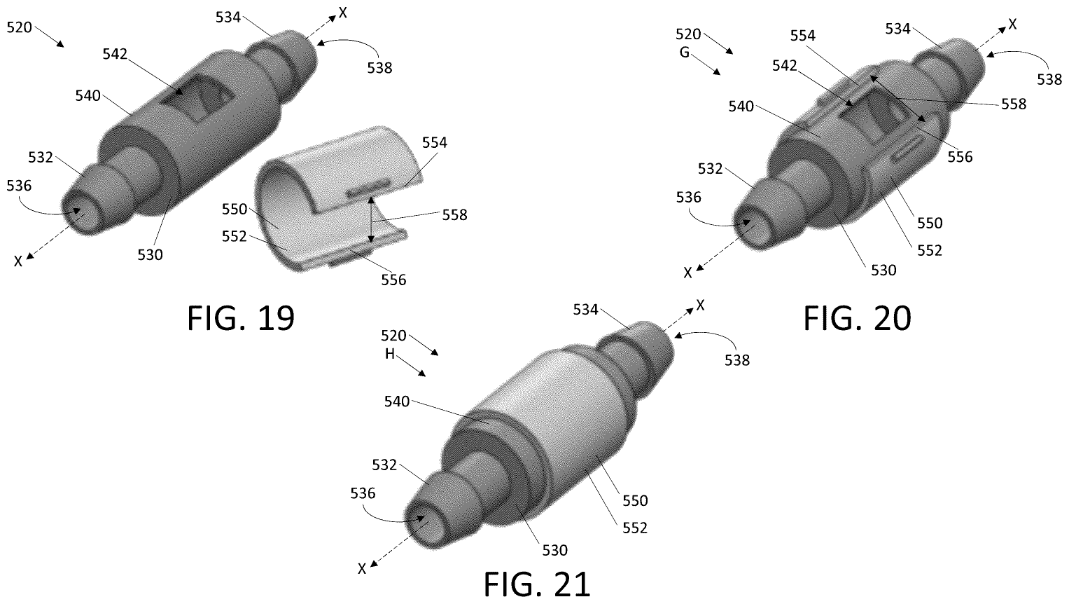

Turning to FIGS. 19-21, and with continued reference to FIGS. 1A-IF and 2-18, illustrated is a rotatable sleeve valve component 520 that may be utilized in a third embodiment of a tubing arrangement 500 that facilitates sterilization of a filter device 100 within a header bag 200. FIGS. 22 and 23 illustrate schematic illustrations of the third embodiment of the tubing arrangement 500 that may be equipped on a filter device 100 and that utilizes the rotatable sleeve valve component 520. The tubing arrangement 500 illustrated in FIGS. 19-23 may be utilized with any type of filter device to facilitate sterilization of that particular filter device. The rotatable sleeve valve component 520 may be utilized in a tubing arrangement 500 that is similar to the tubing arrangements 300, 400 illustrated in FIGS. 5 and 15, respectively. More specifically, the tubing arrangement 500 may be coupled to the filter device 100 via a hose barb connection component 104b installed and disposed on the either the front face 110 or the rear face 111 of the filter device 100 at one of the port openings 108a, 108b, 108c, 108d, 108e, 108f similar to that illustrated in FIGS. 3 and 14. Moreover, also similar to that illustrated in FIGS. 3 and 14, a filter device 100 may be equipped with more than one tubing arrangement 500 (e.g., two, three, etc.). Thus, while FIGS. 22 and 23 illustrate a schematic illustration of a single tubing arrangement 500, the schematic illustrations are for illustrative purposes only, and the illustrated tubing arrangement 500 may be representative of any of the tubing arrangements 500 of the filter device 100. As illustrated in FIGS. 22 and 23, the tubing arrangement 500 may include a tubing 510 that couples one end of the rotatable sleeve valve component 520 to the filter device 100. The tubing arrangement 500 may further include a tubing 512 that couples the opposing end of the rotatable sleeve valve component 520 to an aseptic connector 114a.

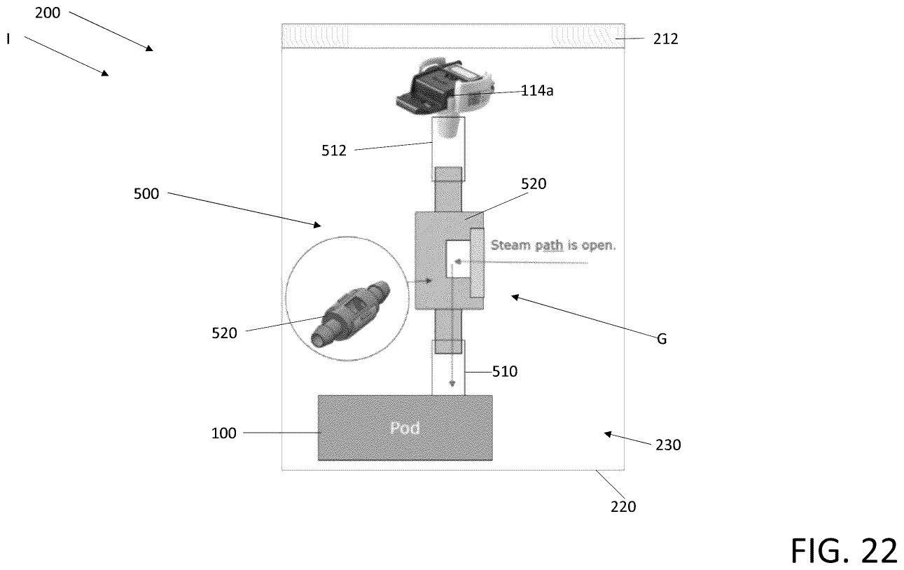

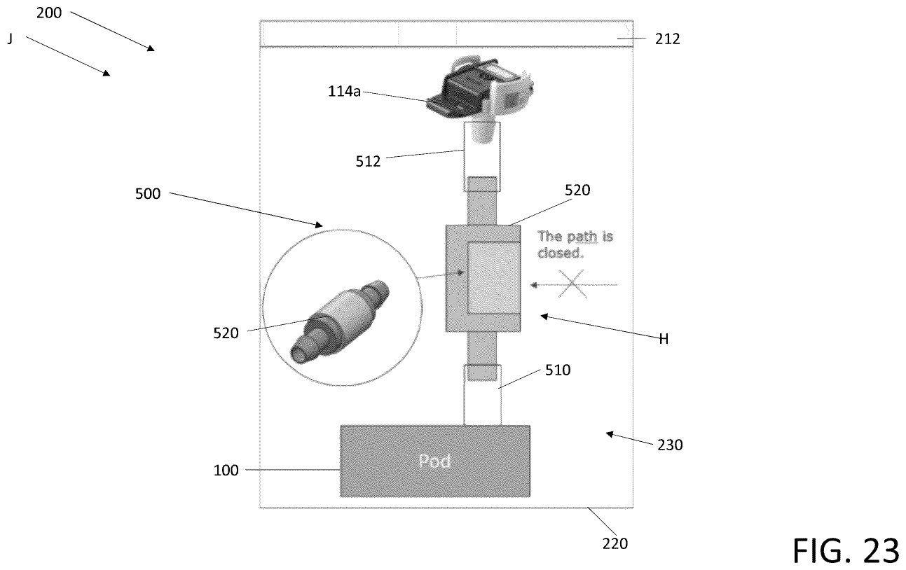

Returning to FIGS. 19-21, illustrated are various views of the rotatable sleeve valve component 520 in various different configurations. More specifically, FIG. 19 depicts the rotatable sleeve valve component 520 in a deconstructed state. FIG. 20 further depicts the rotatable sleeve valve component 520 in the open configuration G, while FIG. 21 depicts the rotatable sleeve valve component 520 in the closed configuration H. As illustrated in FIGS. 19-21, the rotatable sleeve valve component 520 may contain a main portion 530 and a sleeve portion 550. The main portion 530 may be substantially cylindrical with a first connection end 532, an opposite connection end 534, and a central section 540 disposed between the first and second connection ends 532, 534. A longitudinal central axis X may extend centrally through the first connection end 532, the second connection end 534, and the central section 540 such that the first connection end 532, the second connection end 534, and the central section 540 are coaxial with one another. The first connection end 532 may include an aperture 536, while the second connection end 534 may also include an aperture 538. As further illustrated in FIG. 19, the central section 540 includes an opening 542. The apertures 536, 538 and the opening 542 may be in fluid communication with one another.

The sleeve portion 550 may be a substantially annular or ring shaped body 552 that contains a first end 554 and a second end 556 that are spaced from one another by a gap 558. The length of the gap 558 may be wider than that of the opening 542 of the central section 540 of the main portion 530 but smaller than the diameter of the central section 540 of the main portion 530. As illustrated in FIGS. 20 and 21, the sleeve portion 550 may be disposed about the central section 540 of the main portion 530 such that the sleeve portion 550 may slide around the perimeter of the central section 540. In the open configuration G as shown in FIG. 20, the gap 558 of the sleeve portion 550 may be at least partially aligned with the opening 542 of the central section 540 of the main portion 530 so that at least a portion of the opening 542 is exposed. In the closed configuration H shown in FIG. 21, the sleeve portion 550 is rotated about the central section 540 of the main portion 530 so that the opening 542 of the central section 540 of the main portion 530 is fully covered by the body 552 of the sleeve portion 550. The sleeve portion 550 may form a tight enough fit on the central section 540 of the main portion 530 that the sleeve portion 550 is capable of being retained in its position with respect to the central section 540 without freely rotating or sliding around the central section 540. In other words, the sleeve portion 550 may form a tight enough fit on the central section 540 of the main portion 530 that an external force from a user may be required to translate the sleeve portion 550 around the central section 540 of the main portion 530. Moreover, the sleeve portion 550 may form a tight enough fit on the central section 540 of the main portion 530 that the sleeve portion 550 forms a fluid tight seal on the central section 540. Thus, when the opening 542 of the central section 540 is fully covered by the body 552 of the sleeve portion 550, the sleeve portion 550 prevents a fluid from exiting or entering the main portion 530 via the opening 542.

Turning back to FIG. 22, the schematic illustration depicts the modular depth filter device 100 equipped with the third embodiment of the tubing arrangements 500 disposed within the header bag 200 and before the modular depth filter device 100 has been subjected to a sterilization process (e.g., autoclaving and other gas- or vapor-based sterilization methods, such as, but not limited to, ethylene oxide, chlorine dioxide, ozone, supercritical carbon dioxide, and vaporized hydrogen peroxide). As illustrated in FIG. 22, the rotatable sleeve valve component 520 is oriented in the tubing arrangement 500 between the filter device 100 and the aseptic connecter 114a and is in the open configuration G. Thus, the tubing arrangement 500 is in the open state I, where a fluid (e.g., air, vapor, etc.) may flow into and through the tubing arrangement 500 via the opening 542 of the central section 540 of the main portion of the rotatable sleeve valve component 520. Furthermore, when the header bag 200 containing the filter device 100 equipped with the third embodiment of the tubing arrangement 500 is subjected to a sterilization process, a sterilization vapor may pass through the header 212 of the header bag 200 and into the tubing arrangement 500 via the opening 542 of the rotatable sleeve valve component 520. The sterilization vapor may then pass through the rotatable sleeve valve component 520, through the tubing 510, and into the filter device 100 in order to sterilize the filter device 100.

As illustrated in FIG. 23, the rotatable sleeve valve component 520 is in the closed configuration H (i.e., the body 552 of the sleeve portion 550 fully covers the opening 542 of the main portion 530) which changes the tubing arrangement 500 from the open state I to the closed state J. When in the closed state J, a fluid (e.g., air, vapor, etc.) is unable to flow through the opening 542 of the rotatable sleeve valve component 520, into the tubing arrangement 500, and into the filter device 100. Thus, when the rotatable sleeve valve component 520 is in the closed configuration H, the filter device 100 no longer contains a fluid pathway into the interior of the filter device 100.

As further illustrated in FIG. 23, after the header bag 200 and the filter device 100 have been subjected to a sterilization process, and prior to the filter device 100 being removed from the header bag 200, the rotatable sleeve valve components 520 are converted from the open configuration G to the closed configuration H to convert the tubing arrangements 500 from the open state I to the closed state J. More specifically, a user may slide the sleeve portion 550 about the central section 540 of the main portion 530 of the rotatable sleeve valve component 520 such that the body 552 of the sleeve portion 550 fully covers and seals the opening 542 of the main portion 530. Once this has been completed for each of the tubing arrangements 500, the interior of the filter device 100 is isolated from its surrounding environment.

Once the filter device 100 has been sterilized and the tubing arrangements 500 are converted into the closed state J, the filter device 100 may be removed from the interior volume 230 of the header bag 200. Because the rotatable sleeve valve components 520 are in the closed configuration H, the internal sterility of filter device 100 is maintained once the filter device 100 is removed from the header bag 200.

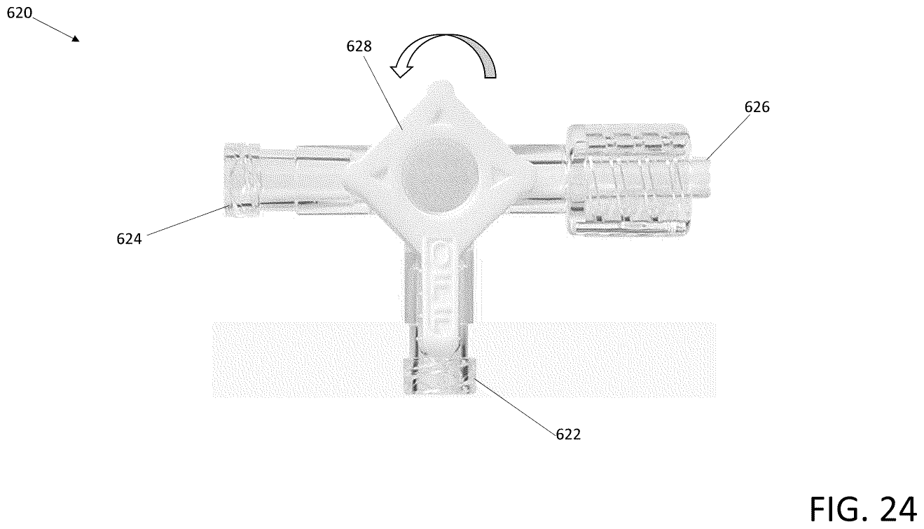

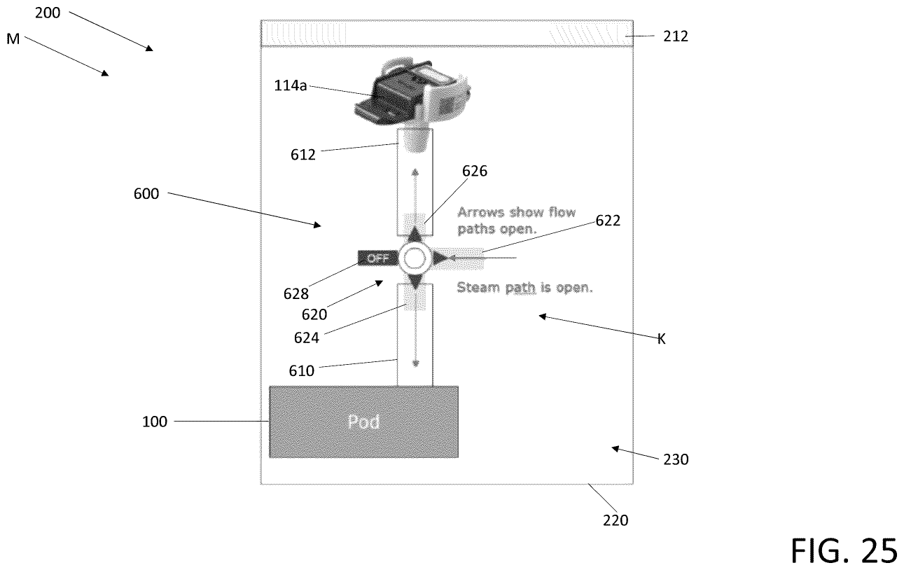

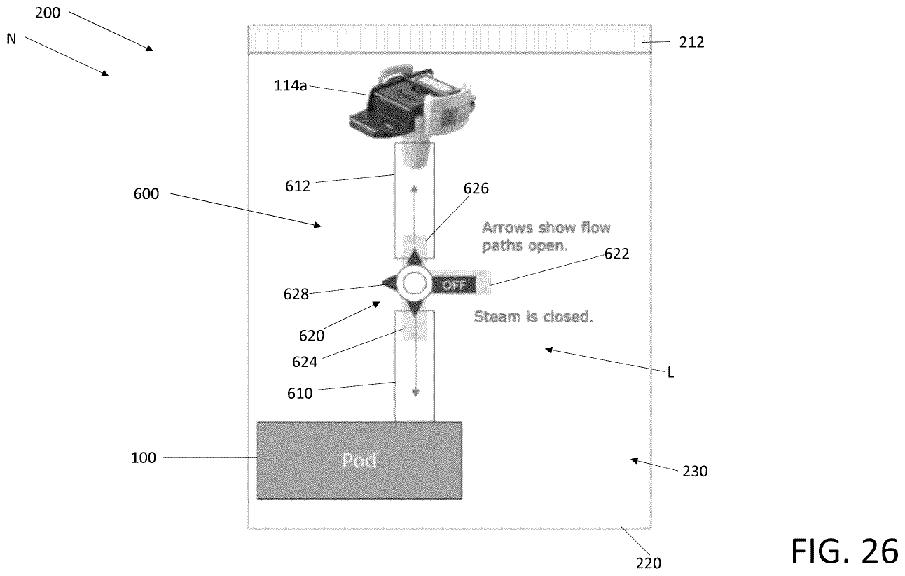

Turning to FIGS. 24-26, and with continued reference to FIGS. 1A-1F and 2-23, illustrated is a 4-way stopcock component 620 that may be utilized in a fourth embodiment of a tubing arrangement 600 that facilitates sterilization of a filter device 100 within a header bag 200. FIG. 24 illustrates an isolated view of the 4-way stopcock component 620, while FIGS. 25 and 26 illustrate schematic illustrations of the fourth embodiment of the tubing arrangement 600 that may be equipped on a filter device 100 and that utilizes the 4-way stopcock component 620. The 4-way stopcock component 620 may be utilized in a tubing arrangement 600 that is similar to the tubing arrangements 300, 400, 500 illustrated in FIGS. 5, 15, and 22, respectively. More specifically, the tubing arrangement 600 may be coupled to the filter device 100 via a hose barb connection component 104b installed and disposed on the either the front face 110 or the rear face 111 of the pod 102 at one of the port openings 108a, 108b, 108c, 108d, 108e, 108f similar to that illustrated in FIGS. 3 and 14. Moreover, also similar to that illustrated in FIGS. 3 and 14, a filter device 100 may be equipped with more than one tubing arrangement 600 (e.g., two, three, etc.). Thus, while FIGS. 25 and 26 illustrate a schematic illustration of a single tubing arrangement 600, the schematic illustrations are for illustrative purposes only, and the illustrated tubing arrangement 600 may be representative of any of the tubing arrangements 600 of the modular depth filter device 100. In addition, the tubing arrangement 600 illustrated in FIGS. 24-26 may be utilized with any type of filter device to facilitate sterilization of that particular filter device.

Returning to FIG. 24, as illustrated, the 4-way stopcock component 620 may include a first connector end 622, a second connector end 624, and a third connector end 626. The second and third connector ends 624, 626 may be longitudinally aligned with one another, while the first connector end 622 is oriented transversely to the second and third connector ends 624, 626. Thus, the connector ends 622, 624, 626 provide the 4-way stopcock 620 with a substantially T-shape. Disposed at the intersection of the connector ends 622, 624, 626 is an actuator 628. The actuator 628 may be rotated with respect to the connector ends 622, 624, 626 to facilitate the opening or closure of each of the connector ends 622, 624, 626. Thus, in other words, certain connector ends 622, 624, 626 may be either opened or closed depending on the rotational position of the actuator 628.

Turning to FIGS. 25 and 26, the tubing arrangement 600 may further include a tubing 610 that couples the second connector end 624 of the 4-way stopcock component 620 to the filter device 100. In addition, the tubing arrangement 600 may also include a tubing 612 that couples the third connector end 626 of the 4-way stopcock component 620 to an aseptic connector 114a. The first connector end 622 may serve as an inlet or outlet to the tubing arrangement 600 depending on the rotational position of the actuator 628.

With specific reference to FIG. 25, illustrated is a schematic illustration that depicts the filter device 100 equipped with the fourth embodiment of the tubing arrangements 600 disposed within the header bag 200 and before the filter device 100 has been subjected to a sterilization process (e.g., autoclaving and other gas- or vapor-based sterilization methods, such as, but not limited to, ethylene oxide, chlorine dioxide, ozone, supercritical carbon dioxide, and vaporized hydrogen peroxide). As illustrated in FIG. 25, the 4-way stopcock component 620 is oriented in the tubing arrangement 600 between the filter device 100 and the aseptic connecter 114a and is in the fully open configuration K. When the 4-way stopcock component 620 is in the fully open configuration K, each of the connector ends 622, 624, 626 are in fluid communication with one another. In other words, when the 4-way stopcock component 620 is in the fully open configuration K, a fluid (e.g., air, vapor, etc.) may enter the 4-way stopcock component 620 via the first connector end 622 and flow into and through both of the second and third connector ends 624, 626 and into the tubing 610, 612. Therefore, when the 4-way stopcock component 620 is in the fully open configuration K, the tubing arrangement 600 is in the open state M, where a fluid (e.g., air, vapor, etc.) may flow into and through the tubing arrangement 600 via the first connector end 622 of the 4-way stopcock component 620. Furthermore, when the header bag 200 containing the filter device 100 equipped with the fourth embodiment of the tubing arrangement 600 is subjected to a sterilization process, a sterilization vapor may pass through the header 212 of the header bag 200 and into the tubing arrangement 600 via the first connector end 622 of the 4-way stopcock component 620. The sterilization vapor may then pass through the second connector end 624 of the 4-way stopcock component 620, through the tubing 610, and into the filter device 100 in order to sterilize the filter device 100.

As illustrated in FIG. 26, the 4-way stopcock component 620 is in the closed configuration L (i.e., the actuator 628 the 4-way stopcock component 620 being rotated such that the first connector end 622 is closed/sealed and a fluid is unable to enter the 4-way stopcock component 620 via the first connector end 622) which changes the tubing arrangement 600 from the open state M to the closed state N. When in the closed state N, a fluid (e.g., air, vapor, etc.) is unable to flow through the first connector end 622 of the 4-way stopcock component 620, into the tubing arrangement 600, and into the filter device 100. Thus, when the 4-way stopcock component 620 is in the closed configuration L, the filter device 100 no longer contains a fluid pathway into the interior of the filter device 100.

As further illustrated in FIG. 26, after the header bag 200 and the filter device 100 have been subjected to a sterilization process, and prior to the filter device 100 being removed from the header bag 200, the 4-way stopcock components 620 are converted from the fully open configuration K to the closed configuration L to convert the tubing arrangements 600 from the open state M to the closed state N. More specifically, a user may slide the actuator 628 about the 4-way stopcock component 620 such that the actuator seals or closes the first connector end 622. Once this has been completed for each of the tubing arrangements 600, the interior of the filter device 100 is isolated from its surrounding environment.

Once the filter device 100 has been sterilized and the tubing arrangements 600 are converted into the closed state N, the filter device 100 may be removed from the interior volume 230 of the header bag 200. Because the 4-way stopcock components 620 are in the closed configuration L, the internal sterility of filter device 100 is maintained once the filter device 100 is removed from the header bag 200.

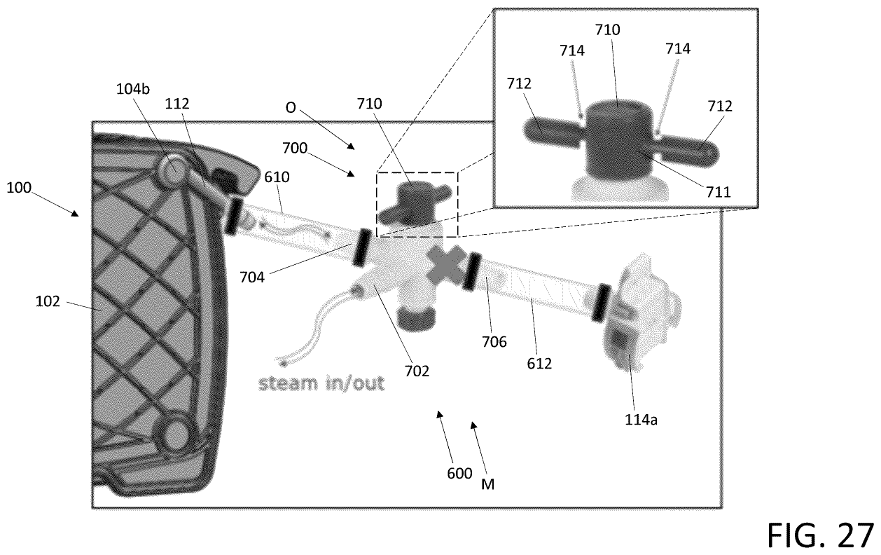

While the tubing arrangement 600 may contain the 4-way stopcock component 620 illustrated in FIGS. 24-26, other variations/embodiments of the stopcock may be utilized that are single-actuation stopcock components. The single-actuation stopcock components 700, 700a, 700b, 700c illustrated in FIGS. 27-29, 30A, and 30B may be utilized in the tubing arrangement 600 in order to maintain the sterility of the filter device 100. As explained in further detail below, the single-actuation stopcock components 700, 700a, 700b, 700c are configured to be operated once and may be a tamper-resistant or tamper-proof once they are oriented in their closed configurations. Thus, the single-actuation stopcock components 700, 700a, 700b, 700c, when utilized in tubing arrangements of the filter device 100 as disclosed herein, prevent contamination of the interior of filter device 100 after it has been rendered sterile.

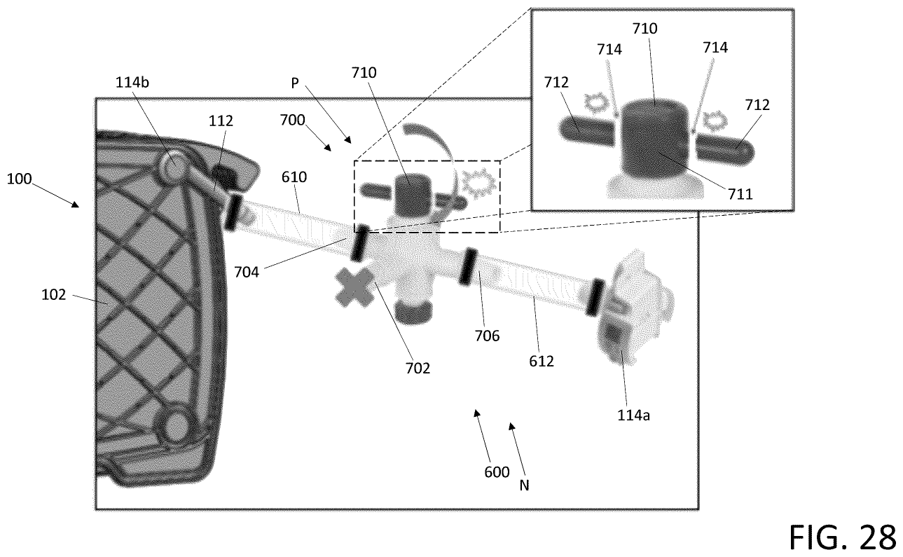

The single-actuation stopcock component 700 illustrated in FIGS. 27 and 28 may contain an actuator 710 with breakaway engagement extensions 712. As illustrated, the single-actuation stopcock component 700 may include a first connector end 702, a second connector end 704, and a third connector end 706. The second and third connector ends 704, 706 may be longitudinally aligned with one another, while the first connector end 702 may be oriented transversely to the second and third connector ends 704, 706. Thus, the connector ends 702, 704, 706 provide the single-actuation stopcock component 700 with a substantial T-shape. As illustrated in FIGS. 27 and 28, the second connector end 704 may be coupled to the hose barb connection component 104b via tubing 610, where the hose barb connection component 104b may be coupled to the filter device 100. As also illustrated, the third connector end 706 may be coupled to the aseptic connector 114a via the tubing 612. The first connector end 702 may remain uncoupled to another device/component, and, thus, may serve as an inlet/outlet of the tubing arrangement 600. Like the 4-way stopcock 620, the single-actuation stopcock component 700 may contain an actuator 710 that is disposed at the intersection of the connector ends 702, 704, 706. The actuator 710 may be rotated with respect to the connector ends 702, 704, 706 to facilitate the opening or closure of each of the connector ends 702, 704, 706. Thus, in other words, certain connector ends 702, 704, 706 may be either opened or closed depending on the rotational position of the actuator 710. Unlike the actuator 628 of the 4-way stopcock component 620, the actuator 710 of the single-actuation stopcock component 700 may include a set of opposing engagement extensions 712 that are coupled to the main body 711 of the actuator 710 via a set of breakaway portions 714. As illustrated, the breakaway portions 714 are thinner in diameter/width than the engagement extensions 712. Thus, the breakaway portions 714 are configured to fail or “break” when the engagement extensions 712 are subjected to a large enough force/torque/pressure that exceeds the yield strength of the material of the engagement extensions 712 and the breakaway portions 714. When the breakaway portions 714 fail, the engagement extensions 712 may break off from the main body 711 of the actuator 710. With the engagement extensions 712 broken off from the main body 711 of the actuator 710, the actuator 710 may become more difficult for a user to engage and rotate the actuator 710. Thus, with the engagement extensions 712 broken off from the main body 711 of the actuator 710, the actuator 710 may be less likely to be inadvertently rotated to an undesired position (i.e., opened). The engagement extensions 712 being broken off from the main body 711 of the actuator 710 may further serve as an indicator that the actuator 710 has already been rotated to a desired (e.g., closed) position.

In the embodiment illustrated in FIG. 27, the single-actuation stopcock component 700 is shown in the open configuration O, while FIG. 28 illustrates the single-actuation stopcock component 700 in the closed configuration P. As illustrated in FIG. 27, when the single-actuation stopcock component 700 is in the open configuration O, the tubing arrangement 600 is in the open state M because a vapor may travel into the tubing arrangement 600 via the first connector end 702, through the single-actuation stopcock component 700, through the second connector end 704, through the tubing 610, and into the filter device 100. However, as illustrated in FIG. 28, when the single-actuation stopcock component 700 is in the closed configuration P, the tubing arrangement 600 is in the closed state N because the single-actuation stopcock component 700 in the closed configuration P may prevent a vapor from entering the tubing arrangement 600 via the first connector end 702. In order to convert the single-actuation stopcock component 700 from the open configuration O to the closed configuration P, the actuator 710 may be rotated with respect to the connector ends 702, 704, 706. As illustrated in FIG. 28, a force may be applied to the engagement extensions 712 of the actuator 710 not only to rotate the actuator 710 from the position in the open configuration O to the position in the closed configuration P, but also to break the engagement extensions 712 off of the main body 711 of the actuator 710 at the breakaway portions 714. In other words, once the actuator 710 has been rotated to the position of the closed configuration P, the actuator 710 may not be capable of further rotation in that direction, but the continued application of the rotational force may cause the engagement extensions 712 to break off of the main body 711 of the actuator 710 at the breakaway portions 714. With the engagement extensions 712 broken off the main body 711 of the actuator 710, it may be more difficult to rotate the actuator 710 from the position in the closed configuration P back to the position in the open configuration O, which further ensures that the filter device 100 is not contaminated after sterilization. In other words, once the engagement extensions 712 have been broken off of the main body 711 of the actuator 710, the single-actuation stopcock component 700 may no longer be tampered with (i.e., the single-actuation stopcock component 700 may not be reconfigured to the open configuration O).

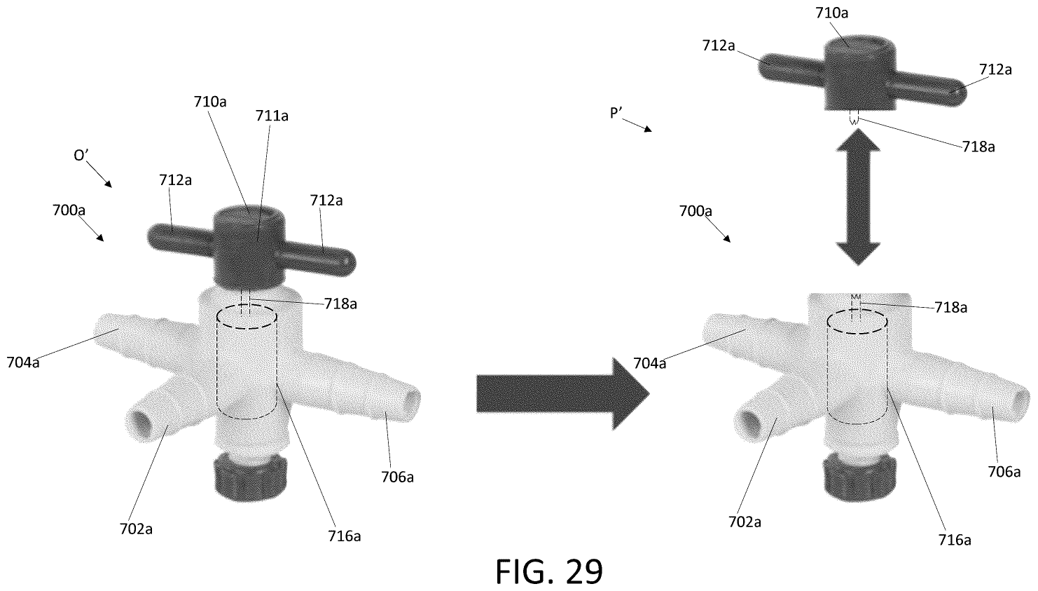

Turning to FIG. 29, illustrated is another single-actuation stopcock component 700a that may be equipped with a mechanism that prevents the actuator 710a from being rotated back to the position in the open configuration O′ from the position in the closed configuration P′. The single-actuation stopcock component 700a illustrated in FIG. 29 may be substantially identical to the single-actuation stopcock component 700 illustrated in FIGS. 27 and 28, except that the engagement extensions 712a of the stopcock 700a may not be connected to the main body 711a via breakaway portions, and the main body 711a of the actuator 710a may be coupled to an internal valve 716a via a breakaway shaft 718a. Thus, when a force is continuously applied to the actuator 710a after the actuator 710a has been rotated from the position of the open configuration O′ to the position of the closed configuration P′, the breakaway shaft 718a may break or fail (i.e., the continuous force may exceed the yield strength of the material of the breakaway shaft 718a). Once the breakaway shaft 718a has failed, the actuator 710a may become disconnected from the internal valve 716a, which prevents the single-actuation stopcock component 700a from being transitioned back to the open configuration O′ (where the first connector end 702a serves as an inlet/outlet to the tubing arrangement 600) from the closed configuration P′. In other words, once the breakaway shaft 718a has failed, the single-actuation stopcock component 700a may be tamper-resistant or tamper-proof, preventing the single-actuation stopcock component 700a from being returned to the open configuration O′. With the single-actuation stopcock component 700a being prevented from transitioning back to the open configuration O′ from the closed configuration P′, sterility of the filter device 100 equipped with the tubing arrangements 600 such that each contain the single-actuation stopcock component 700a may be maintained.

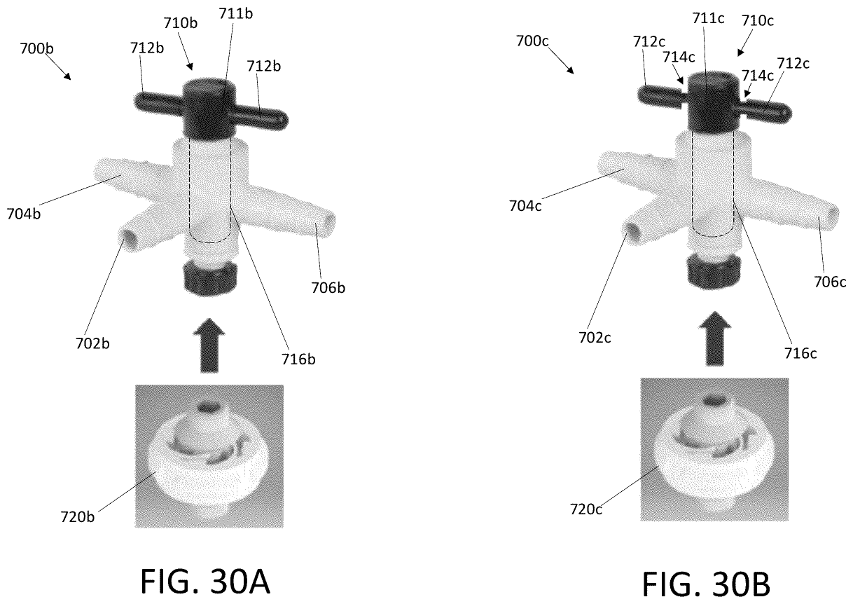

With further regard to FIGS. 30A and 30B, illustrated are additional single-actuation stopcock components 700b, 700c that are equipped with mechanisms that prevent the single-actuation stopcock components 700b, 700c from being converted back to the open configurations from the closed configurations. Both of the single-actuation stopcock components 700b, 700c may be utilized in a tubing arrangement like that explained above with regard to the 4-way stopcock components 620 and the single-actuation stopcock components 700, 700a in order to both sterilize a filter device 100 and maintain the sterility of the filter device 100. The single-actuation stopcock component 700b illustrated in FIG. 30A may be substantially similar to the single-actuation stopcock component 700 except that the single-actuation stopcock component 700b does not contain breakaway portions that couple the engagement extensions 712b to the main body 711b of the actuator 710b, but does include a ratchet mechanism 720b disposed within the interior of the single-actuation stopcock component 700b and coupled, either directly or indirectly (e.g., via the interior valve 716b), to the actuator 710b. The ratchet mechanism 720b may be configured to enable the actuator 710b to be rotated in a first direction (i.e., which allows the actuator 710b of the single-actuation stopcock component 700b to rotate from the position in the open configuration to the position in the closed configuration) and prevents the actuator 710b from rotating in a second direction (i.e., prevents the actuator 710b of the single-actuation stopcock component 700b from rotating from the position in the closed configuration back to the position in the open configuration). Thus, the ratchet mechanism 720b prevents the single-actuation stopcock component 700b from being converted out of the closed configuration, which, when the single-actuation stopcock component 700b forms part of the tubing arrangement of a filter device 100, maintains the sterility of the filter device 100.

The single-actuation stopcock component 700c illustrated in FIG. 30B may be substantially similar to the single-actuation stopcock component 700b illustrated in FIG. 30A except that the single-actuation stopcock component 700c may contain breakaway portions 714c that couple the engagement extensions 712c to the main body 711c of the actuator 710c. Thus, similar to that of the single-actuation stopcock component 700b illustrated in FIG. 30A, the single-actuation stopcock component 700c may include a ratchet mechanism 720c disposed within the interior of the single-actuation stopcock component 700c and coupled, either directly or indirectly (e.g., via the interior valve 716c), to the actuator 710c. As previously explained with regard to the ratchet mechanism 720b, the ratchet mechanism 720c may be configured to enable the actuator 710c to be rotated in a first direction (i.e., which allows the actuator 710c of the single-actuation stopcock component 700c to rotate from the position in the open configuration to the position in the closed configuration) and prevents the actuator 710c from rotating in a second direction (i.e., prevents the actuator 710c of the single-actuation stopcock component 700c from rotating from the position in the closed configuration back to the position in the open configuration). The breakaway portions 714c, as previously explained, may be configured to break or “fail” when the force applied to the engagement extensions 712c exceeds the yield strength of the material in which the engagement extensions 712c and the breakaway portions 714c are constructed. Thus, the combination of the breakaway portions 714c and ratchet mechanism 720b prevents the single-actuation stopcock component 700c from being converted out of the closed configuration, which, when the single-actuation stopcock component 700c forms part of the tubing arrangement of a filter device 100, maintains the sterility of the filter device 100.

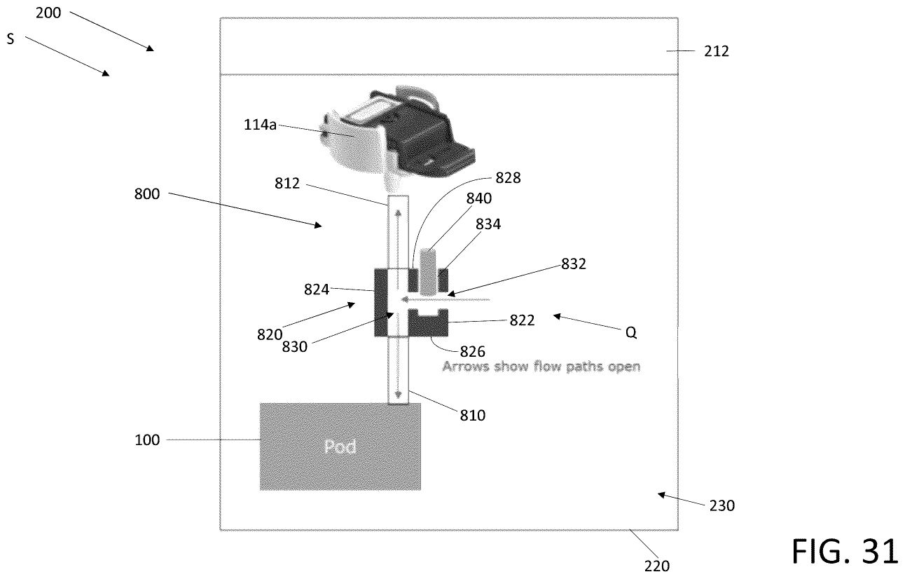

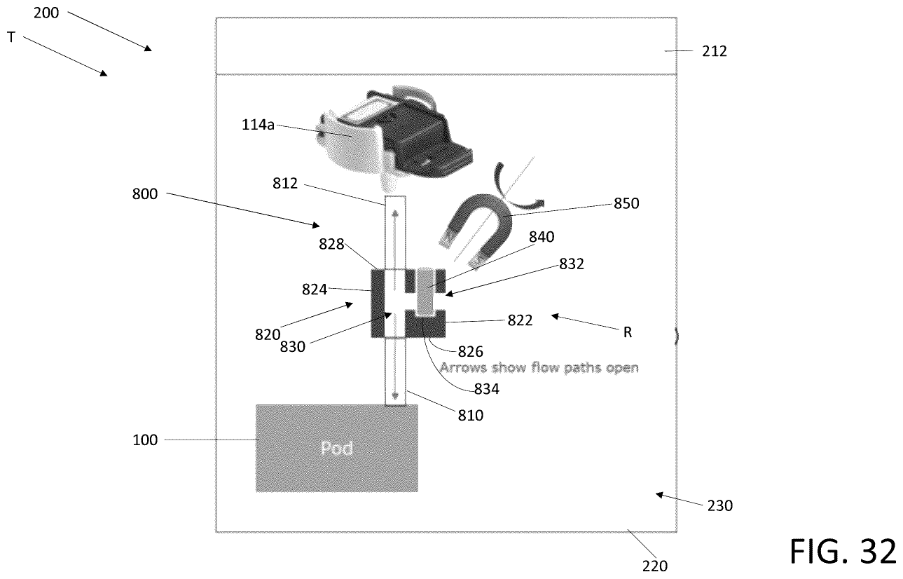

Turning to FIGS. 31 and 32, and with continued reference to FIGS. 1A-1F and 2-29, 30A, and 30B, illustrated is a schematic illustration of a magnetic valve component 820 that may be utilized in a fifth embodiment of a tubing arrangement 800 that facilitates sterilization of a filter device 100 within a header bag 200. The magnetic valve component 820 may be utilized in a tubing arrangement 800 that is similar to the tubing arrangements 300, 400, 500, 600 illustrated in FIGS. 5, 15, 22, and 25 respectively. More specifically, the tubing arrangement 800 may be coupled to the filter device 100 via a hose barb connection component 104b installed and disposed on the either the front face 110 or the rear face 111 of the pod 102 at one of the port openings 108a, 108b, 108c, 108d, 108e, 108f similar to that illustrated in FIGS. 3 and 14. Moreover, also similar to that illustrated in FIGS. 3 and 14, a filter device 100 may be equipped with more than one tubing arrangement 800 (e.g., two, three, etc.). Thus, while FIGS. 31 and 32 illustrate a schematic illustration of a single tubing arrangement 800, the schematic illustrations are for illustrative purposes only, and the illustrated tubing arrangement 800 may be representative of any of the tubing arrangements 800 of the modular depth filter device 100. Furthermore, the tubing arrangement 800 illustrated in FIGS. 31 and 32 may be utilized with any type of filter device to facilitate sterilization of that particular filter device.

As further illustrated in FIGS. 31 and 32, the tubing arrangement 800 may include a tubing 810 that couples the magnetic valve component 820 to the filter device 100. In addition, the tubing arrangement 800 may also include a tubing 812 that couples the magnetic valve component 820 to an aseptic connector 114a.

The magnetic valve component 820 may include a first side 822, an opposite second side 824, a third side 826 spanning between the first side 822 and the second side 824, and a fourth side 828 opposite the third side 826 that also spans between the first side 822 and the second side 824. While the magnetic valve component 820 may be illustrated as having a square cross-section, the magnetic valve component 820 may be of any other shape that is capable of being reconfigured between an open configuration and a closed configuration as described below. As further illustrated, the magnetic valve component 820 may include an elongated passageway 830 that extends through the magnetic valve component 820 between the third side 826 and the fourth side 828. The tubing 810 fluidly coupling the magnetic valve component 820 with the filter device 100 may be coupled to the third side 826 of the magnetic valve component 820 at one end of the elongated passageway 830. The tubing 812 fluidly coupling the magnetic valve component 820 with the aseptic connector 114a may be coupled to the fourth side 828 of the magnetic valve component 820 at the opposite end of the elongated passageway 830 from that of tubing 810.

As further illustrated in FIGS. 31 and 32, the magnetic valve component 820 may further include an intermediate passageway 832 that extends into the magnetic valve component 820 via the first side 822 of the magnetic valve component 820 and that intersects the elongated passageway 830. The magnetic valve component 820 may also include a slot 834 that is spaced from the elongated passageway 830, but intersects the intermediate passageway 832. Slidably, rotationally, or repositonably disposed within the slot 834 is magnetic closure element 840. The magnetic closure element 840 may be oriented in an open position Q (as illustrated in FIG. 31) or a closed position R (as illustrated in FIG. 32) depending on a magnetic force applied to the magnetic valve component 820 by a magnet 850. In some embodiments, the magnetic closure element 840 may include a magnetic threaded rod (not shown) that may be magnetically driven to translate the magnetic closure element 840 into the closed position R by means of a rotating magnetic field (e.g., from a rotating tool that generates a rotating magnetic field).

With specific reference to FIG. 31, illustrated is a schematic illustration that depicts the filter device 100 equipped with the fifth embodiment of the tubing arrangements 800 disposed within the header bag 200 and before the filter device 100 has been subjected to a sterilization process (e.g., autoclaving and other gas- or vapor-based sterilization methods, such as, but not limited to, ethylene oxide, chlorine dioxide, ozone, supercritical carbon dioxide, and vaporized hydrogen peroxide). As illustrated in FIG. 31, the magnetic valve component 820 is oriented in the tubing arrangement 800 between the filter device 100 and the aseptic connector 114a and the magnetic closure element 840 is in the open position Q. When the magnetic closure element 840 is in the open position Q, the elongated passageway 830 and the intermediate passageway 832 are in fluid communication with one another. In other words, when the magnetic closure element 840 is in the open position Q, a fluid (e.g., air, vapor, etc.) may enter the magnetic valve component 820 at the first side 822 via the intermediate passageway 832, flow into and through the elongated passageway 830, and flow into and through the tubing 810, 812. Thus, when the magnetic closure element 840 is in the open configuration Q, the tubing arrangement 800 is in the open state S, where a fluid (e.g., air, vapor, etc.) may flow into and through the tubing arrangement 800 at the first side 822 of the magnetic valve component 820 via the intermediate passageway 832. Furthermore, when the header bag 200 containing the filter device 100 equipped with the fifth embodiment of the tubing arrangement 800 is subjected to a sterilization process, a sterilization vapor may pass through the header 212 of the header bag 200 and into the tubing arrangement 800 via the intermediate passageway 832 of the magnetic valve component 820. The sterilization vapor may then pass through the intermediate passageway 832, into and through elongated passageway 830, through the tubing 810, and into the filter device 100 in order to sterilize the filter device 100.

As illustrated in FIG. 32, the magnetic closure element 840 of the magnetic valve component 820 is in the closed position R (i.e., a magnet 850 has applied a magnetic force to the magnetic closure element 840 to translate the magnetic closure element 840 along the slot 834 from the open position Q to the closed position R), where the magnetic closure element 840 has closed or sealed at least a portion of the intermediate passageway 832 from the elongated passageway 830. When the magnetic closure element 840 is in the closed position R, the tubing arrangement 800 is in the closed state T because the magnetic closure element 840 sealing the intermediate passageway 832 prevents a fluid from flowing through the magnetic valve component 820. In other words, when in the tubing arrangement 800 is in the closed state T, a fluid (e.g., air, vapor, etc.) is unable to flow through the intermediate passageway 832 of the magnetic valve component 820, into the elongated passageway 830, and into the filter device 100. Thus, when the magnetic closure element 840 is in the closed configuration R, the filter device 100 no longer contains a fluid pathway into the interior of the filter device 100.

As further illustrated in FIG. 32, after the header bag 200 and the filter device 100 have been subjected to a sterilization process, and prior to the modular depth filter device 100 being removed from the header bag 200, the magnetic valve components 820 are subjected to a magnetic force from a magnet 850 (e.g., via a rotating tool configured to generate a rotating magnetic field) to convert the magnetic closure elements 840 from the open position Q to the closed position R to convert the tubing arrangements 800 from the open state S to the closed state T. Once this has been completed for each of the tubing arrangements 800, the interior of the filter device 100 is isolated from its surrounding environment. Once the filter device 100 has been sterilized and the tubing arrangements 800 are converted into the closed state T, the filter device 100 may be removed from the interior volume 230 of the header bag 200. Because the magnetic closure elements 840 of the magnetic valve components 820 are in the closed position R, the sterility of filter device 100 is maintained once the filter device 100 is removed from the header bag 200. The use of the magnetic valve component 820 may simplify the process of sealing the filter device 100 while the filter device 100 remains within the header bag 200 because it eliminates the need to physically manipulate the valve component through the header bag 200 and decreases the chance the head bag 200 is pierced.

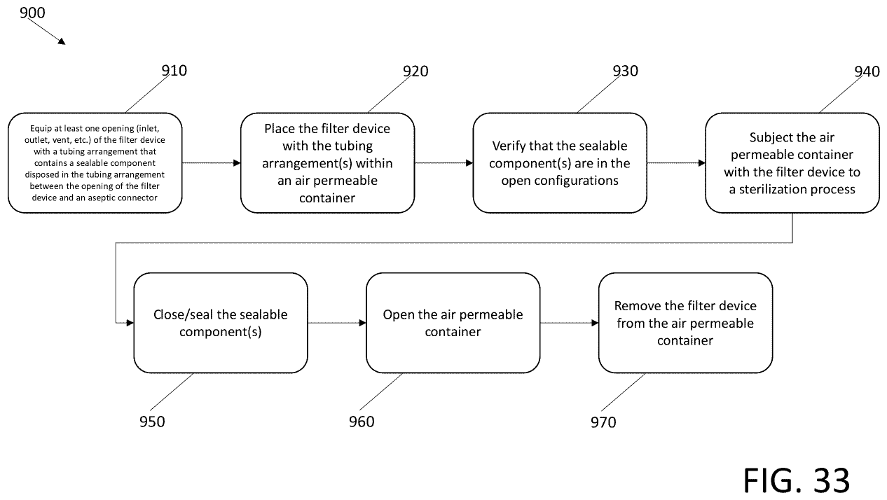

Turning to FIG. 33, illustrated is a flowchart 900 depicting the process for sterilizing a filter device (e.g., modular depth filter device 100) equipped with one or more tubing arrangements 300, 400, 500, 600, 800 that contain a sealable component (e.g., spring actuated valve component 330, pinch clamp 430, rotatable sleeve valve component 520, stopcock components 620, 700, 700a, 700b, 700c, magnetic valve component 820, etc.) to retain the sterility of the filter device after the sterilization process is complete and the filter device is removed from the sterilization container (e.g., header bag 200). At 910, the filter device is equipped with at least one tubing arrangement 300, 400, 500, 600, 800 at an opening of the filter device. The filter device may be any type of filter device including, but not limited to, the modular depth filter device 100 depicted herein. The tubing arrangement(s) equipped onto the filter device may be any type of tubing arrangement including, but not limited to, the tubing arrangements 300, 400, 500, 600, 800 depicted herein. In addition, should the filter device be equipped with more than one tubing arrangement, the tubing arrangements may all be the same as one another, or may be different types of tubing arrangements so long as each of the tubing arrangements contains a sealable component (e.g., spring actuated valve component 330, pinch clamp 430, rotatable sleeve valve component 520, stopcock components 620, 700, 700a, 700b, 700c, magnetic valve component 820, etc.) depicted herein. The tubing arrangements may be equipped onto the filter device at any type of opening on the filter device, including, but not limited to, an inlet, an outlet, a vent opening, etc. At 920, once the filter device is equipped with a tubing arrangement with a sealable component, the filter device is placed within a container having at least one breathable microbial barrier. While the header bag 200 illustrated in FIG. 2 depicts one embodiment of a container having at least one breathable microbial barrier (i.e., header 212), any container having a breathable microbial barrier (i.e., that is configured to enable air, vapor, etc. to pass through the barrier while preventing any microbials from passing through the barrier) like that of the header bag 200 may be used in step 920. At 930, while the filter device is disposed within the container, a user may verify that the sealable component(s) are in the open configuration or open state. As an example, if the tubing arrangement was equipped with valve components 330, a user may verify that a female portion 350 is coupled to its respective male portion 340. As another example, if the tubing arrangement was equipped with the pinch clamp 430, a user may verify the pinch clamp 430 is in the unclamped position C.

Once the user verifies that the sealable component(s) are in the open configuration or states, the user, at 940, subjects the container housing the filter device to a sterilization process. As previously explained, the sterilization process includes subjecting the container and, because of the breathable microbial barrier of the container, the filter device disposed within the container, to autoclaving and/or other gas- or vapor-based sterilization methods, such as, but not limited to, ethylene oxide, chlorine dioxide, ozone, supercritical carbon dioxide, and vaporized hydrogen peroxide. As previously explained, any sterilization vapor may pass through the breathable microbial barrier of the container and through the tubing arrangement(s) of the filter device with the tubing arrangement(s) being in the open configuration. At 950, once the sterilization process is complete, and prior to opening the container and removing the filter device, the user may close or seal the sealable components. For example, when the container is the header bag 200 and the sealable component is the valve component 330, the user may manipulate the valve component 330 through the header bag 200 in order to detach the female portion 350 from the male portion 340, which seals the tubing arrangement 300 and places it in the closed state B. In another example, when the container is the header bag 200 and the sealable component is the pinch clamp 430, the user may manipulate the pinch clamp 430 through the header bag 200 in order to reconfigure the pinch clamp 430 from the unclamped position C to the clamped position D, which seals the tubing arrangement 400 and places it in the closed state F.

With the filter device in the container sterilized and the tubing arrangement(s) in the closed state, the container with the breathable microbial barrier may be opened at 960. At 970, the user may remove the filter device from the container. As explained above regarding the various tubing arrangements 300, 400, 500, 600, 800 and the sealable components (e.g., spring actuated valve component 330, pinch clamp 430, rotatable sleeve valve component 520, stopcock components 620, 700, 700a, 700b, 700c, magnetic valve component 820, etc.), with the sealable component(s) sealing the tubing arrangement(s) and retaining the tubing arrangement(s) in the closed state, the sterility of the filter device, and particularly the interior of the filter device, is maintained despite being out of the container. The sterility of the filter device may be maintained so long as the sealable component(s) of the tubing arrangement(s) are retaining the tubing arrangement(s) in the closed state.

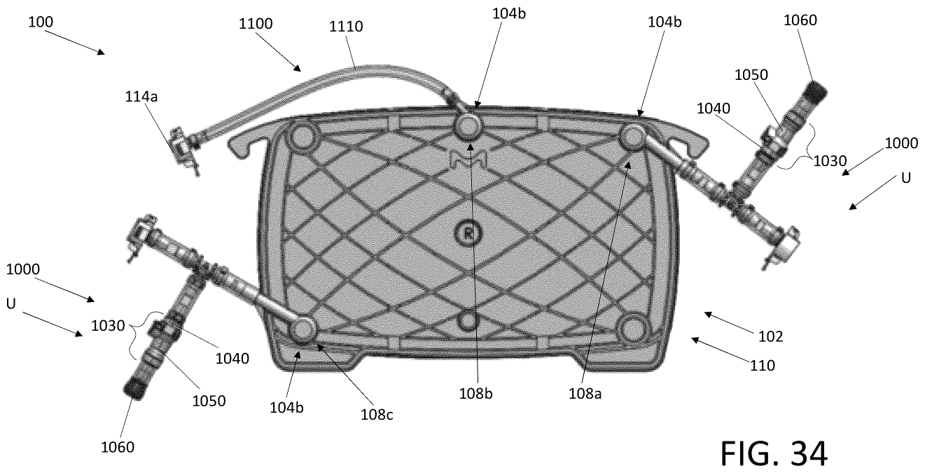

Turning to FIGS. 34-38, and with continued reference to FIGS. 1A-1F and 2-11, illustrated is a modular depth filter device 100 equipped with another embodiment of a tubing arrangement 1000 that facilitates sterilization of the modular depth filter device 100 without the use of the header bag 200 or other container equipped with a breathable microbial barrier. While a modular depth filter device 100 is illustrated, the modular depth filter device 100 is intended as an example only. The tubing arrangement 1000, 1100 illustrated in FIGS. 34-38 may be utilized with any other type of filter device to facilitate sterilization of that particular filter device. As best illustrated in FIG. 34, hose barb connection components 104b are installed and disposed on the front face 110 of the pod 102 of the modular depth filter device 100 at the port openings 108a, 108b, 108c. As further illustrated, tubing arrangements 1000 may be coupled to the hose barb connection components 104b at the inlet 108a and the outlet 108b. A different tubing arrangement 1100 may be coupled to the hose barb connection component 104b at the vent 108b via tubing 1110. The tubing 1110 may enable the aseptic connector 114a to be in fluid communication with the vent 108b. While not illustrated, and as previously explained, blind end components 104a may be installed and disposed on the rear face 111 of the pod 102 of the modular depth filter device 100 at port openings 108d, 108e, 108f, which are in fluid communication and opposite to the port openings 108a, 108b, 108c, respectively, disposed on the front face 110. While the modular depth filter device 100 is depicted as being equipped with two tubing arrangements 1000, 1100, the modular depth filter device 100 may also be equipped with only one type of tubing arrangements 1000, or with three different tubing arrangements. Moreover, the tubing arrangements 1000, 1100 may be equipped on different port openings 108a, 108b, 108c, 108d, 108e, 108f than that shown in FIG. 34.

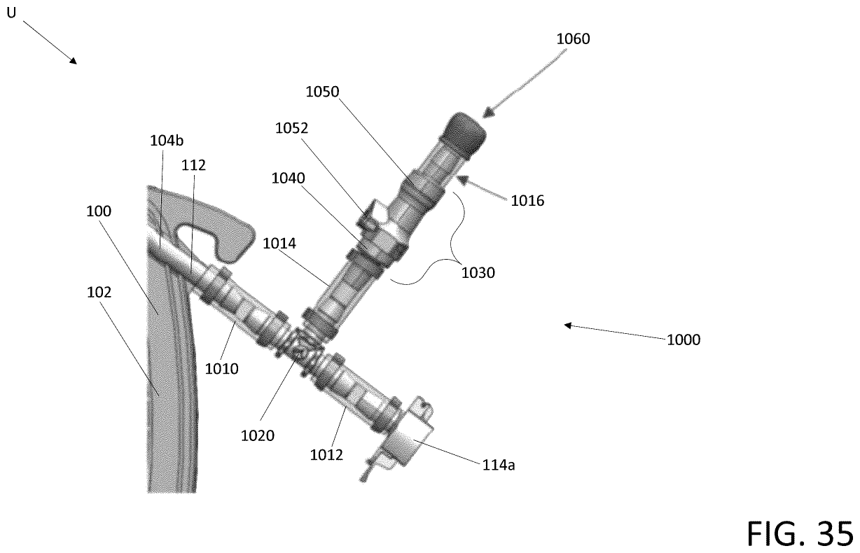

FIGS. 34 and 35 depict the tubing arrangement 1000 of the filter device 100 in the open state U. The tubing arrangement 1000, as explained in further detail below, enables the filter device 100 to be subjected to a sterilization process while retaining the sterilization of the modular depth filter device 100 after completion of the sterilization process. While FIG. 35 only depicts a single tubing arrangement 1000, the tubing arrangement 1000 may represent any of the tubing arrangements 1000 illustrated in FIG. 34. The tubing arrangement 1000 may include a series of tubing 1010, 1012, 1014, 1016, where the tubing 1010, 1012, 1014 are connected to one another via a tee fitting 1020. As further illustrated, tubing 1010 may be connected between the hose barb fitting 112 of the hose barb connection component 104b and the tee fitting 1020. Tubing 1012 may be connected between an aseptic connector 114a and the tee fitting 1020. Furthermore, tubing 1014 may be connected between a valve component 1030 and the tee fitting 1020. The valve component 1030 may be a spring actuated valve component having a male portion 1040 and a female portion 1050 that are capable of being disconnected from one another. In FIGS. 34 and 35, the male portion 1040 is shown inserted into, and coupled to, the female portion 1050. The female portion 1050 may further include an actuator 1052 that, when actuated, may facilitate the disconnection or uncoupling of the female portion 1050 from the male portion 1040. As further illustrated, the tubing 1014 may be connected between the tee fitting 1020 and end of the male portion 1040 of the valve component 1030 that is opposite of the end of the male portion 1040 configured to couple to the female portion 1050. The tubing 1016 may be connected to the end of the female portion 1050 of the valve component 1030 that is opposite of the end of the female portion 1050 configured to couple to the male portion 1040.

FIGS. 34 and 35 further illustrate that a breathable microbial barrier 1060 is disposed on an end of the tubing 1016 that is opposite of the end of the tubing 1016 that is coupled to the female portion 1050 of the valve component 1030. The breathable microbial barrier 1060 may be constructed from high-density polyethylene fibers that are either interwoven or flashspun to create a material that is vapor permeable (e.g., water vapor, etc.) while also being capable of withstanding gamma irradiation or ethylene oxide gas that is used to sterilize equipment and serving as a microbial barrier. The breathable microbial barrier 1060 may also be puncture-resistant and durable. In some embodiments, the breathable microbial barrier 1060 may be equipped with an elasticized band to retain the breathable microbial barrier 1060 on the end of the tubing 1016. As explained below, the vapor permeability of the breathable microbial barrier 1060 allows the modular depth filter device 100 to be subjected to a sterilization process (e.g., autoclaving, ethylene oxide, chlorine dioxide, ozone, supercritical carbon dioxide, and vaporized hydrogen peroxide, etc.) by allowing a sterilization vapor or gas to enter and pass through the tubing arrangement 1000 and the depth filter device 100. In addition, the high-density polyethylene of the breathable microbial barrier 1060 enables the tubing arrangement 1000 and the interior of the filter device 100 to remain sterile after being subjected to the sterilization process and prior to closure of the valve component 1030. Thus, the breathable microbial barrier 1060 allows for steam penetration, air removal, and drying of the tubing arrangement 1000 and the filter device 100 while also serving as a microbial barrier.

As best illustrated in FIGS. 34 and 35, the valve component 1030 is in the open state U. When the female portion 1050 of the valve component 1030 is coupled to the male portion 1040, the valve component 1030 is in the open state U. In the open state U, a fluid (e.g., air, vapor, etc.) may flow through the breathable microbial barrier 1060, through the tubing 1016, through the valve component 1030, and into the filter device 100 via tubing 1014, the tee fitting 1020, tubing 1010, and the hose barb connector component 104b. With the female portion 1050 of the valve component 1030 of each tubing arrangement 1000 coupled to the respective male portion 1040, each tubing arrangement 1000 of the modular depth filter device 100 provides a fluid pathway into the filter device 100. Because the breathable microbial barrier 1060 is disposed over the inlet end of the tubing 1016, which is coupled to the female portion 1050, coupling the female portion 1050 to the male portion 1040 of the valve component 1030 also couples the breathable microbial barrier 1060 to the tubing arrangement 1000. In other words, when the valve component 1030 is in the open state U, the breathable microbial barrier 1060 forms a type of seal on the inlet of the tubing arrangement 1000 that enables the passage of a fluid (e.g., air, vapor, etc.) into the tubing arrangement 1000 and the filter device 100 while resisting the passage of microbes.

With the valve components 1030 in the open state U, and the breathable microbial barriers 1060 being disposed on the inlets to the tubing arrangements 1000, the filter device 100 may then be subjected to a sterilization process (e.g., autoclaving and other gas- or vapor-based sterilization methods, such as, but not limited to, ethylene oxide, chlorine dioxide, ozone, supercritical carbon dioxide, and vaporized hydrogen peroxide) where a sterilizing vapor may flow into the interior of the filter device 100 via the tubing arrangements 1000. As previously explained, when the female portion 1050 is connected to the male portion 1040, the tubing arrangement 1000 provides an open pathway for the sterilizing vapor to flow into the filter device 100. More specifically, the sterilization vapor may flow into the tubing arrangement 1000 via the breathable microbial barrier 1060 and the valve component 1030, through the tubing 1014, through the tee fitting 1020, and through the tubing 1010 into the filter device 100.

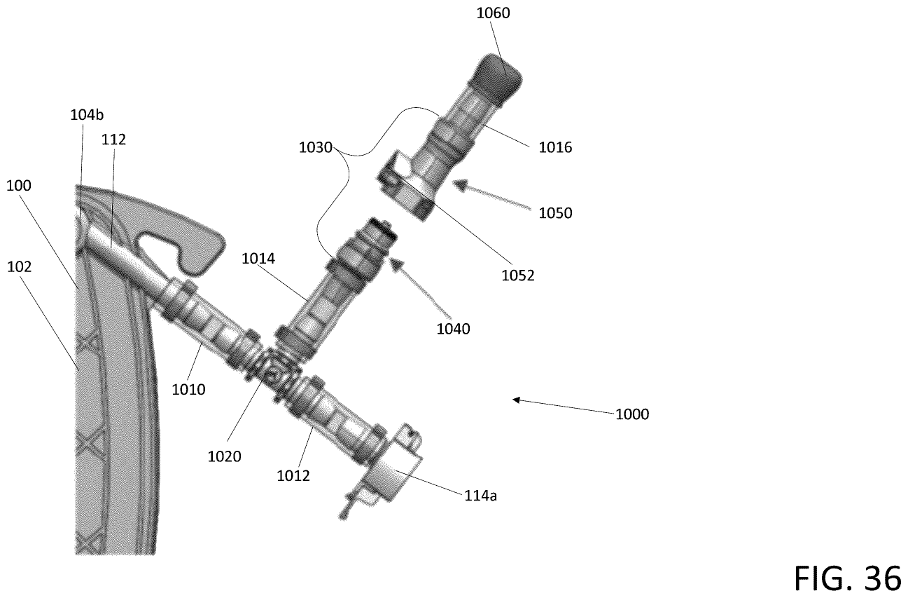

FIG. 36 illustrates the valve component 1030 of the tubing arrangement 1000 being transitioned from in the open state U to the closed state V. The male portion 1040 of the valve component 1030 may be equipped with a spring actuated valve (not shown) that is biased to the closed position. When the female portion 1050 is coupled to the male portion 1040 of the valve component 1030, however, the spring actuated valve is oriented in the open position by the connection of the female portion 1050 to the male portion 1040. Thus, as explained previously, when the female portion 1050 is coupled to the male portion 1040, the valve component 1030 is in the open state U. With the spring actuated valve being biased to the closed position, when the female portion 1050 is removed or disconnected from the male portion 1040, the spring actuated valve repositions from the open position to the closed position. Thus, uncoupling the female portion 1050 from the male portion 1040 places the valve component 1030 in the closed state V. As further illustrated in FIG. 36, because the breathable microbial barrier 1060 is disposed over the inlet end of the tubing 1016, which is coupled to the female portion 1050, the uncoupling of the female portion 1050 from the male portion 1040 also uncouples the tubing 1016 and the breathable microbial barrier 1060 from the male portion 1040 and from the tubing arrangement 1000.

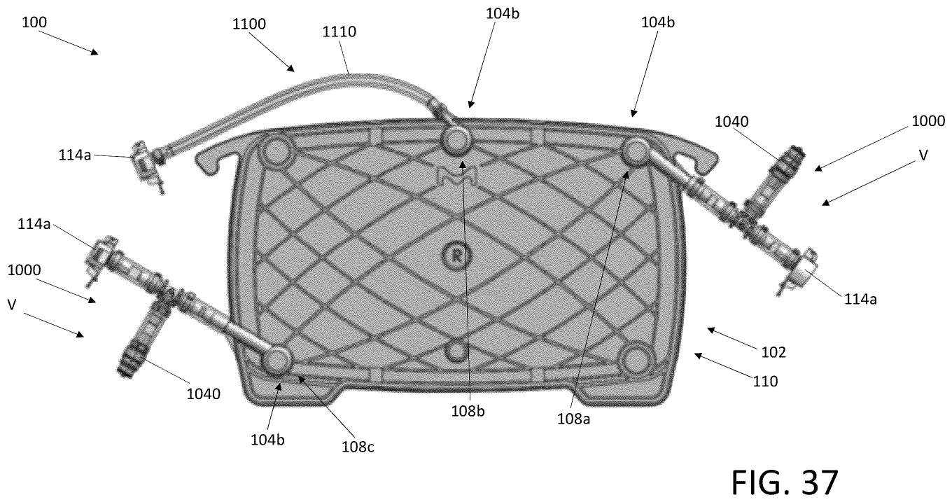

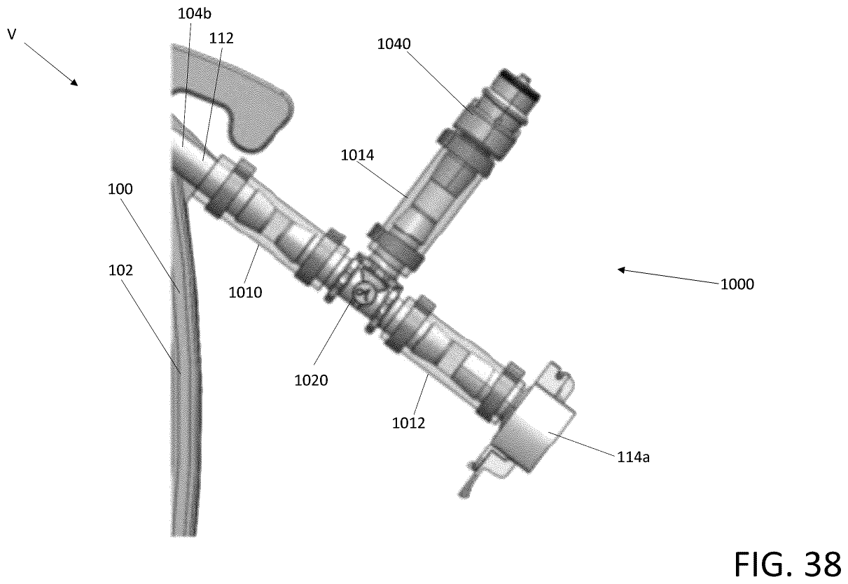

As best illustrated in FIGS. 37 and 38, the valve component 1030 is in the closed state V due to the female portion 1050 of the valve component 1030 being uncoupled or disconnected from the male portion 1040. When the valve component 1030 is in the closed state V, as previously explained, the spring actuated valve is in the closed state due to the spring actuated valve being biased to the closed state and due to the absence of the female portion 1050 that, when coupled to the male portion 1040, forces the spring actuated valve to the open position. Therefore, when the valve component 1030 is in the closed state V, a fluid (e.g., air, vapor, etc.) is unable to flow through the valve component 1030 and into the filter device 100. In other words, when the female portion 1050 of the valve component 1030 of each tubing arrangement 1000 is uncoupled/disconnected from the respective male portion 1040, the filter device 100 no longer contains a fluid pathway into the interior of the filter device 100.

The female portion 1050 of the valve component 1030 of each of the tubing arrangements 1000 is removed from its respective male portion 1040 in order to convert the tubing arrangements 1000 from the open state U to the closed state V after the filter device 100 has been subjected to a sterilization process (e.g., autoclaving and/or other gas- or vapor-based sterilization methods, such as, but not limited to, ethylene oxide, chlorine dioxide, ozone, supercritical carbon dioxide, and vaporized hydrogen peroxide). Returning to FIG. 36 the female portion 1050 may be removed from the male portion 1040 of the valve component 1030 via the actuator 1052. More specifically, the actuator 1052 of the female portion 1050 may be actuated (e.g., depressed, slid in one direction, etc.) in order to facilitate the removal or disconnection of the female portion 1050 from the male portion 1040. Because, as previously explained, the male portion 1040 of each of the valve components 1030 contains a spring actuated valve that is biased closed when the respective female portion 1050 is disconnected, the sterility of the filter device 100 is maintained once the tubing arrangements 1000 are converted into the closed state V.

While FIGS. 34-36 illustrate a breathable microbial barrier 1060 disposed on a tubing arrangement 1000 equipped with the spring actuated valve component 1030, the embodiment utilizing the breathable microbial barrier 1060 instead of the header bag 200 may be utilized with any type of sealable valve component, including, but not limited to, the pinch clamp 430, the rotatable sleeve valve component 520, the stopcock components 620, 700, 700a, 700b, 700c, and the magnetic valve component 820.

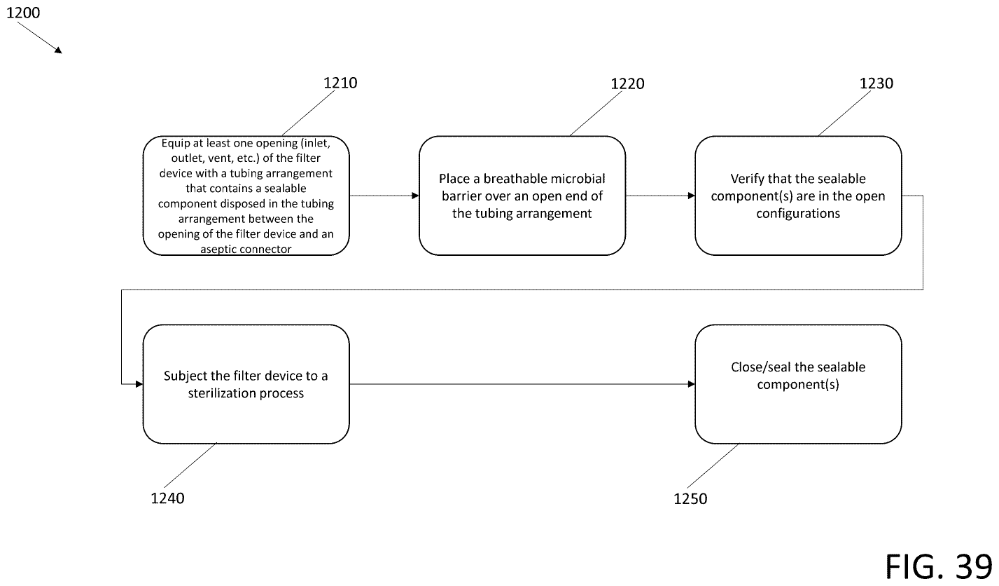

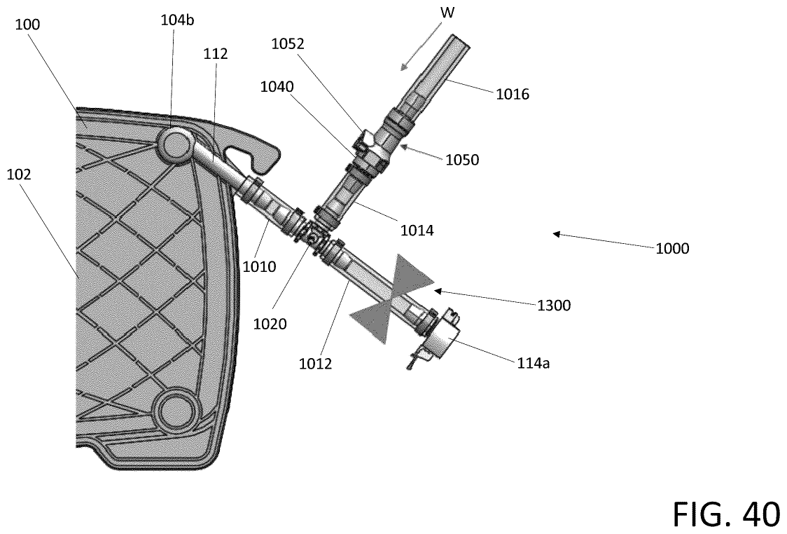

Turning to FIG. 39, illustrated is a flowchart 1200 depicting the process for sterilizing a filter device (e.g., modular depth filter device 100) equipped with one or more tubing arrangements 1000 that contain a sealable component (e.g., spring actuated valve component 1030, etc.) and a breathable microbial barrier 1060 disposed over each inlet of the tubing arrangements 1000. At 1210, the filter device is equipped with at least one tubing arrangement 1000 at an opening of the filter device. The filter device may be any type of filter device including, but not limited to, the modular depth filter device 100 depicted herein. The tubing arrangement(s) equipped onto the filter device may be any type of tubing arrangement including, but not limited to, the tubing arrangement 1000 depicted herein. In addition, should the filter device be equipped with more than one tubing arrangement, the tubing arrangements may all be the same as one another, or may be different types of tubing arrangements so long as each of the tubing arrangements contain a sealable component (e.g., spring actuated valve component 1030, etc.) as depicted herein. The tubing arrangements may be equipped onto the filter device at any type of opening on the filter device, including, but not limited to, an inlet, an outlet, a vent opening, etc. At 1220, once the filter device is equipped with a tubing arrangement with a sealable component, a breathable microbial barrier 1060 is disposed on each opening (e.g., inlet, outlet, etc.) of the tubing arrangements. As previously explained, the breathable microbial barrier 1060 may be configured to enable air, vapor, etc. to pass through the barrier while preventing or resisting any microbes from passing through the barrier. At 1230, a user may verify that the sealable component(s) are in the open configuration or open state. As an example, if the tubing arrangement was equipped with valve components 1030, a user may verify that the female portion 1050 is coupled to the male portion 1040.

Once the user verifies that the sealable component(s) are in the open configuration or states, the user, at 1240, subjects the filter device to a sterilization process. As previously explained, the sterilization process includes subjecting the filter device to autoclaving and/or other gas- or vapor-based sterilization methods, such as, but not limited to, ethylene oxide, chlorine dioxide, ozone, supercritical carbon dioxide, and vaporized hydrogen peroxide. As previously explained, any sterilization vapor may pass through the breathable microbial barrier 1060 disposed on an opening of the tubing arrangement(s) of the filter device with the tubing arrangement(s) being in the open configuration. At 1250, once the sterilization process is complete, the user may close or seal the sealable components. For example, when the sealable component is the valve component 1030, the user may manipulate (e.g., depress the actuator 1052) the valve component 1030 to detach the female portion 1050 and the breathable microbial barrier 1060 from the male portion 1040, which seals the tubing arrangement 1000 and places it in the closed state V. With the filter device sterilized and the tubing arrangement(s) in the closed state, the sterility of the filter device, and particularly the interior of the filter device, is maintained. The sterility of the filter device may be maintained so long as the sealable component(s) of the tubing arrangement(s) are retained in the closed state.

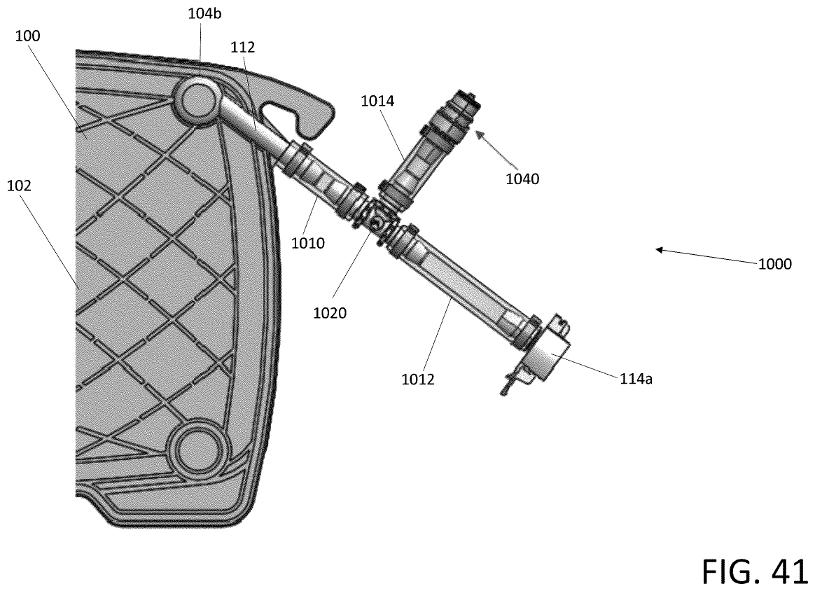

Turning to FIGS. 40 and 41, illustrated is the tubing arrangement 1000 without the breathable microbial barrier 1060 that facilitates in-process leakage integrity testing when the device is sterilized. While FIGS. 40 and 41 illustrate a single tubing arrangement 1000 of the filter device 100 (e.g., disposed at the inlet of the filter device 100), the method/process described herein may be applicable to the other tubing arrangements 1000 (e.g., disposed at the vent and the outlet of the filter device 100) of the filter device 100. Moreover, while a modular depth filter device 100 is illustrated, the modular depth filter device 100 is intended as an example only. The tubing arrangement 1000 illustrated in FIGS. 40 and 41 may be utilized with any other type of filter device to facilitate sterilization of that particular filter device.

After the tubing arrangement has been constructed and coupled to the filter device 100, there is a need to check whether there are any leaks present in the tubing arrangements 1000 (e.g., welded, joined, fastened, or clamped regions such as where clamps or zip ties are used to fasten/secure tubing to various hose barb connection components 104). The method/process described here may be used to test for leakage integrity of the tubing arrangement 1000 and the filter device 100 except for the joint between the tubing 1012 and the aseptic connector 114a due to the presence of a breathable membrane in the aseptic connector 114a, which causes inherent leakage. Thus, if a tee fitting 1020 is in place in the tubing arrangement 1000 coupled to a filter device 100, a clamping device 1300 (i.e., represented by red triangles in FIG. 40) may be placed onto the tubing 1012 between the tee fitting 1020 and the aseptic connector 114a in order to isolate the aseptic connector 114a and due to the inherent leakage of the breathable microbial membrane disposed within the aseptic connector 114a. While FIG. 40 illustrates the clamping device 1300 as a schematic object, any known clamping device, including, but not limited to, the pinch clamp 430 disclosed herein, may be utilized to clamp the tubing 1012. Once the tubing 1012 is clamped with the clamping device 1300, pressurized air W may be introduced through the inlet/port of the tubing 1016. A leakage, if any, in the tubing arrangement 1000 may be detected by performing a pressure decay leak test with a pressure gauge (not shown) placed at the inlet/port of the tubing 1016 (i.e., where the pressurized air W is introduced into the tubing arrangement 1000). Once this leak test is complete, the clamping device 1300 may be removed from the tubing 1012 and female portion 1050 of the valve component 1030 may be disconnected from the male portion 1040 of the valve component 1030 as shown in FIG. 41.

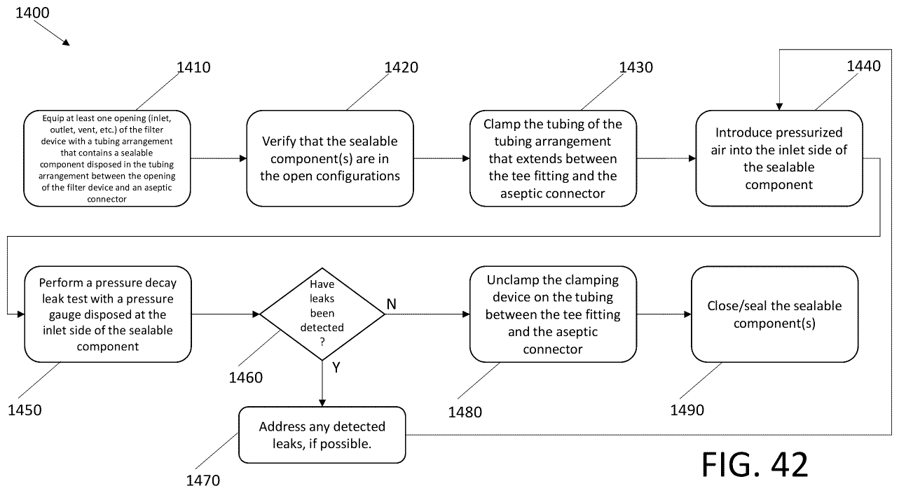

Illustrated in FIG. 42 is a flowchart 1400 depicting the process for testing for leaks in a filter device (e.g., modular depth filter device 100) and the one or more tubing arrangements 1000 disposed on the filter device 100. At 1410, the filter device may be equipped with at least one tubing arrangement 1000 at an opening (e.g., inlet, outlet, vent, etc.) of the filter device. The filter device may be any type of filter device including, but not limited to, the modular depth filter device 100 depicted herein. The tubing arrangement(s) equipped onto the filter device may be any type of tubing arrangement including, but not limited to, the tubing arrangement 1000 depicted herein. In addition, should the filter device be equipped with more than one tubing arrangement, the tubing arrangements may all be the same as one another, or may be different types of tubing arrangements so long as each of the tubing arrangements contain a sealable component (e.g., spring actuated valve components 330, 1030, pinch clamp 430, rotatable sleeve valve component 520, stopcock components 620, 700, 700a, 700b, 700c, magnetic valve component 820, etc.) as depicted herein. The tubing arrangements may be equipped onto the filter device at any type of opening on the filter device, including, but not limited to, an inlet, an outlet, a vent opening, etc. At 1420, once the filter device is equipped with a tubing arrangement with a sealable component, a user may verify that the sealable component(s) are in the open configuration or open state. As an example, if the tubing arrangement was equipped with valve components 1030, a user may verify that the female portion 1050 is coupled to the male portion 1040.

At 1430, should the tubing arrangement be equipped with a tee fitting, like that of the tubing arrangement 1000, which is equipped with tee fitting 1020, the user may then clamp the tubing of a tubing arrangement between the tee fitting and the aseptic connector with a clamping device. With the tubing clamped, the user may then, at 1440, introduced pressurized air into the inlet tubing arrangement via the inlet side of the sealable component of the tubing arrangement. At 1450, a pressure decay leak test with a pressure gauge (not shown) placed at the inlet side of the sealable component (e.g., where the pressurized air is introduced into the tubing arrangement) may be performed.

At 1460, a determination is made on whether or not leaks are present in the tubing arrangement and/or the filter device based on the results of the pressure decay leak test results. If, at 1460, leaks are detected, then, at 1470, the user may address any detected leaks. This includes, but is not limited to, addressing any of the welded, joined, fastened, or clamped regions of the tubing arrangement. Once the user has addressed the leaks at 1470, the process/method returns to step 1440 in order to perform steps 1440 and 1450 and see if the leaks have been adequately addressed. Steps 1440, 1450, 1460, and 1470 may be continuously performed until the leaks are no longer detected.

If, at 1460, no leaks are detected based on the pressure decay test results, then, at 1480, a user may unclamp the clamping device that is disposed on the tubing proximate to the aseptic connector. At 1490, a user may close or seal the sealable component of the tubing arrangement. For example, when the sealable component is the valve component 1030, the user may manipulate (e.g., depress the actuator 1052) the valve component 1030 to detach the female portion 1050 and the tubing 1016, which contains the inlet of the tubing arrangement, from the male portion 1040. As previously explained, because the male portion 1040 of the valve component 1030 contains a spring actuated valve biased to the closed position, when the female portion 1050 is disconnected from the male portion 1040, the valve component 1030 is changed to the closed state V, which seals the tubing arrangement 1000.

While the apparatuses presented herein have been illustrated and described in detail and with reference to specific embodiments thereof, it is nevertheless not intended to be limited to the details shown, since it will be apparent that various modifications (e.g., various different types of filter devices) and structural changes may be made therein without departing from the scope of the inventions and within the scope and range of equivalents of the claims.

In addition, various features from one of the embodiments may be incorporated into another of the embodiments. That is, it is believed that the disclosure set forth above encompasses multiple distinct inventions with independent utility. While each of these inventions has been disclosed in a preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Accordingly, it is appropriate that the appended claims be construed broadly and in a manner consistent with the scope of the disclosure as set forth in the following claims.

It is also to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment of the invention. Additionally, it is also to be understood that the components of the apparatuses described herein, the heat extraction assembly described herein, or portions thereof may be fabricated from any suitable material or combination of materials, such as, but not limited to, plastic or metals (e.g., copper, bronze, aluminum, steel, etc.), as well as derivatives thereof, and combinations thereof. In addition, it is further to be understood that the steps of the methods described herein may be performed in any order or in any suitable manner.

Finally, when used herein, the term “comprises” and its derivations (such as “comprising”, etc.) should not be understood in an excluding sense, that is, these terms should not be interpreted as excluding the possibility that what is described and defined may include further elements, steps, etc. Similarly, where any description recites “a” or “a first” element or the equivalent thereof, such disclosure should be understood to include incorporation of one or more such elements, neither requiring nor excluding two or more such elements. Meanwhile, when used herein, the term “approximately” and terms of its family (such as “approximate”, etc.) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies, etc. The same applies to the terms “about”, “around”, “generally”, and “substantially.”

What is claimed is:

1. A tubing arrangement for sterilizing or bioburden-reducing a filter device, the tubing arrangement comprising:
a first tube coupled to a connector of the filter device;
a second tube coupled to an aseptic connector; and
a sealable component disposed in the tubing arrangement between the first and second tubes, wherein the sealable component is reconfigurable between an open configuration, which facilitates entry of a sterilization vapor into the tubing arrangement and the filter device when subjected to a sterilization or bioburden-reduction process, and a closed configuration, where the tubing arrangement is sealed from a surrounding environment.

2. The tubing arrangement of claim 1, further comprising:
a tee fitting having a first end, a second end, and a third end, the first end being coupled to the first tube and the second end being coupled to the second tube.

3. The tubing arrangement of claim 2, wherein the sealable component is coupled to the third end of the tee fitting and the sealable component is a spring actuated valve component comprising:
a male portion; and
a female portion removably coupled to the male portion, wherein when the female portion is coupled to the male portion, the sealable component is in the open configuration, and when the female portion is uncoupled from the male portion, the sealable component is in the closed configuration.

4. The tubing arrangement of claim 2, wherein a third tubing is coupled to the third end of the tee fitting and the sealable component is a pinch clamp disposed on the third tubing.

5. The tubing arrangement of claim 1, wherein the sealable component is a stopcock coupled to both the first tube and the second tube.

6. The tubing arrangement of claim 1, wherein the sealable component is a rotatable sleeve valve component.

7. The tubing arrangement of claim 1, wherein the sealable component is a magnetic valve component.

8. A method for sterilizing or bioburden-reducing a filter device, the method comprising:
equipping at least one opening of the filter device with a tubing arrangement, wherein the tubing arrangement includes an aseptic connector, a sealable component disposed in the tubing arrangement between the at least one opening of the filter device and the aseptic connector, and a port;
enclosing at least the port of the tubing arrangement with a breathable microbial barrier;
subjecting the filter device to a sterilization or bioburden-reduction process; and
converting the sealable component of the tubing arrangement from an open configuration to a closed configuration.

9. The method of claim 8, wherein the sterilization or bioburden-reduction process is autoclaving.

10. The method of claim 8, wherein the sterilization or bioburden-reduction process is a gas- or vapor-based sterilization process.

11. The method of claim 8, wherein the breathable microbial barrier includes an elasticized band that enables the breathable microbial barrier to be disposed over the port of the tubing arrangement.

12. The method of claim 8, wherein the breathable microbial barrier is a portion of a container that defines an interior volume.

13. The method of claim 12, wherein enclosing at least the port of the tubing arrangement within the breathable microbial barrier further comprises:
sealing the filter device and the tubing arrangement within the interior volume of the container.

14. The method of claim 13, wherein the sealable component of the tubing arrangement are converted to the closed configuration while the filter device is sealed within the container.

15. The method of claim 12, wherein the container is a header bag and the breathable microbial barrier is formed as a header of the header bag.

16. A system for sterilizing or bioburden-reducing a filter device, the system comprising:
a filter device having at least one opening; and
a tubing arrangement coupled to the at least one opening of the filter device, the tubing arrangement comprising:
an aseptic connector;
a port in fluid communication with the aseptic connector; and
a sealable component disposed in the tubing arrangement between the at least one opening of the filter device and the aseptic connector, the sealable component being in fluid communication with the at least one opening of the filter device, the aseptic connector, and the port, wherein the sealable component is reconfigurable between an open configuration, which facilitates entry of a sterilization vapor into the tubing arrangement and the filter device when subjected to a sterilization or bioburden-reduction process, and a closed configuration, where the tubing arrangement is sealed from a surrounding environment; and
a breathable microbial barrier at least partially enclosing the port of the tubing arrangement.

17. The system of claim 16, wherein the breathable microbial barrier is incorporated into a header of a header bag that defines an interior volume, wherein the filter device and the tubing arrangement are disposed within the interior volume of the header bag.

18. The system of claim 16, wherein the breathable microbial barrier includes an elasticized band that enables the breathable microbial barrier to be disposed over the port of the tubing arrangement.

19. The system of claim 16, wherein the tubing arrangement further comprises:
a tee fitting having a first end, a second end, and a third end;
a first tubing connecting the first end of the tee fitting to the at least one opening of the filter device;
a second tubing connecting the second end of the tee fitting to the aseptic connector; and
a third tubing coupled to the third end of the tee fitting.

20. The system of claim 19, wherein the sealable component is a spring actuated valve component comprising:
a male portion; and
a female portion removably coupled to the male portion, wherein, when the female portion is coupled to the male portion, the sealable component is in the open configuration, and when the female portion is uncoupled from the male portion, the sealable component is in the closed configuration.
